(19) United States
(12) Patent Application Publication
(10) Pub. No.: US 2016/0120474 A1
(43) Pub. Date: May 5, 2016

(54) Wearable Device for the Ear with Electroencephalographic and Spectroscopic Sensors

(71) Applicant: Robert A. Connor, Forest Lake, MN (US)

(72) Inventor: Robert A. Connor, Forest Lake, MN (US)

(73) Assignee: Medibotics LLC, Forest Lake, MN (US)

(21) Appl. No.: 14/992,073

(22) Filed: Jan. 11, 2016

Related U.S. Application Data
(63) Continuation-in-part of application No. 14/599,522, filed on Jan. 18, 2015.
Application No. 14/599,522 is a continuation-in-part of application No. 14/562,719, filed on Dec. 7, 2014.
Application No. 14/562,719 is a continuation-in-part of application No. 13/523,739, filed on Jun. 14, 2012, now Pat. No. 9,042,596.
Application No. 13/523,739 is a continuation-in-part of application No. 14/550,953, filed on Nov. 22, 2014.
Application No. 14/550,953 is a continuation-in-part of application No. 13/523,739, filed on Jun. 14, 2012, now Pat. No. 9,042,596.
Application No. 13/523,739 is a continuation-in-part of application No. 13/616,238, filed on Sep. 14, 2012.
Application No. 13/616,238 is a continuation-in-part of application No. 13/616,238, filed on Sep. 14, 2012.
(60) Provisional application No. 61/932,517, filed on Jan. 28, 2014.
(60) Provisional application No. 61/932,517, filed on Jan. 28, 2014.
(60) Provisional application No. 61/939,244, filed on Feb. 12, 2014.
(60) Provisional application No. 62/017,615, filed on Jun. 26, 2014.
(60) Provisional application No. 62/089,696, filed on Dec. 9, 2014.
(60) Provisional application No. 61/932,517, filed on Jan. 28, 2014.

(51) Int. Cl. A61B 5/00; A61B 5/0478; A61B 7/04

(52) U.S. Cl. CPC A61B 5/6817; A61B 7/04; A61B 5/0075; A61B 5/7405; A61B 5/0478; A61B 5/72; A61B 5/683; A61B 2560/0214

(57) ABSTRACT

This invention is a wearable device for the ear comprising: an ear attachment with a forehead-projecting portion, an electromagnetic energy sensor which is configured to collect data concerning electromagnetic energy which is transmitted through and/or emitted by the person's body, a microphone, a spectroscopic sensor which is configured to collect data concerning light energy which is transmitted through and/or reflected from the person's body, a sound-emitting unit, a power source, and a data processor. This device configuration is advantageous for mobile electroencephalographic monitoring and spectroscopic analysis of body tissue.

### CROSS-REFERENCE TO RELATED APPLICATIONS

This patent application:

(1) is a continuation-in-part of U.S. patent application Ser. No. 14/599,522 entitled “Mobile Wearable Electromagnetic Brain Activity Monitor” by Robert A. Connor filed on Jan. 18, 2015 which, in turn—(a) is a continuation-in-part of U.S. patent application Ser. No. 14/562,719 entitled “Willpower Glasses™ —A Wearable Food Consumption Monitor” by Robert A. Connor with a filing date of Dec. 7, 2014 which was a continuation-in-part of U.S. patent application Ser. No. 13/523,739 entitled “The Willpower Watch™: A Wearable Food Consumption Monitor” by Robert A. Connor with a filing date of Jun. 14, 2012 and also claimed the priority benefit of U.S. Provisional Patent Application No. 61/932,517 entitled “Nutrode™: Wearable EEG Monitor for Modifying Food Consumption” by Robert A. Connor with a filing date of Jan. 28, 2014; (b) claims the priority benefit of U.S. Provisional Patent Application No. 61/932,517 entitled “Nutrode™: Wearable EEG Monitor for Modifying Food Consumption” by Robert A. Connor with a filing date of Jan. 28, 2014; (c) claims the priority benefit of U.S. Provisional Patent Application No. 61/939,244 entitled “Brainwave-Controlled Eyewear” by Robert A. Connor with a filing date of Feb. 12, 2014; (d) claims the priority benefit of U.S. Provisional Patent Application No. 62/017,615 entitled “Nervision™ Integrated Eyewear and EEG Monitor” by Robert A. Connor with a filing date of Jun. 26, 2014; and (e) claims the priority benefit of U.S. Provisional Patent Application No. 62/089,696 entitled “Electroencephalographic Eyewear” by Robert A. Connor with a filing date of Dec. 9, 2014;

(2) is a continuation-in-part of U.S. patent application Ser. No. 14/550,953 entitled “Wearable Food Consumption Monitor” by Robert A. Connor filed on Nov. 22, 2014 which, in turn—(a) is a continuation-in-part of U.S. patent application Ser. No. 13/523,739 entitled “The Willpower Watch™: A Wearable Food Consumption Monitor” by Robert A. Connor filed on Jun. 14, 2012; (b) is a continuation-in-part of U.S. patent application Ser. No. 13/616,238 entitled “Interactive Voluntary and Involuntary Caloric Intake Monitor” by Robert A. Connor filed on Sep. 14, 2012; and (c) claims the priority benefit of U.S. Provisional Patent Application No. 61/932,517 entitled “Nutrode™: Wearable EEG Monitor for Modifying Food Consumption” by Robert A. Connor filed on Jan. 28, 2014; and

(3) is a continuation-in-part of U.S. patent application Ser. No. 13/616,238 entitled “Interactive Voluntary and Involuntary Caloric Intake Monitor” by Robert A. Connor filed on Sep. 14, 2012.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to mobile devices for monitoring electromagnetic brain activity and for spectroscopic analysis of body tissue.

### Introduction

There are many potential applications for measurement of electromagnetic brain activity (e.g. measuring electroencephalographic EEG activity) and for spectroscopic analysis of body tissue (e.g. measuring changes in the chemistry of blood or interstitial fluid). One such application is monitoring and measuring food consumption. Another application is the use of electromagnetic brain activity generally as a Human-to-Computer Interface (HCl). For such applications, it is desirable to have a mobile wearable device which enables measurement of a person's electromagnetic brain activity and spectroscopic analysis of their body tissue while the person goes about their normal daily activities.

There are mobile electroencephalographic monitors in the prior art, including some which span a portion of a person's forehead, but they tend to be rather obtrusive in appearance. They are not the sort of devices that most people would wish to wear as they go about their normal daily activities. There are also mobile spectroscopic sensors in the prior art, including some worn on the finger tip or wrist. However, most people would not wish to wear a device on their finger tip for an extended period of time. Also, the human wrist is so thick that it can be difficult to transmit light through it for spectroscopic analysis.

The ear is a promising location for a device which can measure electromagnetic brain activity (e.g. from a sensor on a forward-projecting arm which extends toward a person's forehead) and can also conduct spectroscopic analysis of body tissue (e.g. from a sensor which measures light transmitted through the person's earlobe). The invention disclosed herein is a mobile device with both electroencephalographic and spectroscopic sensors which is attached to the ear. This mobile device can be used for monitoring and measuring food consumption. It can also be used as a general Human-to-Computer Interface (HCl).

### Review of the Prior Art

The prior art includes some devices with electromagnetic energy sensors worn on or in the ear which can measure electromagnetic brain activity. Most of these devices are similar in configuration to a conventional hearing aid or ear bud. However, conventional hearing aid and ear bud designs do not include forward projections which extend to a portion of person's forehead. Thus, they are not well-suited for holding an electromagnetic energy sensor in contact with a person's forehead when that is a desired location for collecting brain activity data. Further, conventional hearing aid and ear bud designs are not well-suited for spectroscopic analysis of light transmitted through a person's earlobe.

Relevant prior art concerning devices with electromagnetic energy sensors which are worn on or in the ear includes U.S. Pat. No. 6,694,180 (Boesen, Feb. 17, 2004, “Wireless Biopotential Sensing Device and Method with Capability of Short-Range Radio Frequency Transmission and Reception”), U.S. Pat. No. 8,157,730 (Leboeuf et al., Apr. 17, 2012, “Physiological and Environmental Monitoring Systems and Methods”), U.S. Pat. No. 8,204,786 (LeBoeuf et al., Jun. 19, 2012, “Physiological and Environmental Monitoring Systems and Methods”); and applications 20060094974 (Cain, May 4, 2006, “Systems and Methods for Detecting Brain Waves”), 20070112277 (Fischer et al., May 17, 2007, “Apparatus and Method for the Measurement and Monitoring of Bioelectric Signal Patterns”), 20080146890 (LeBoeuf et al., Jun. 19, 2008, “Telemetric Apparatus for Health and Environmental Monitoring”), and 20080146892 (LeBoeuf et al., Jun. 19, 2008, “Physiological and Environmental Monitoring Systems and Methods”).

This relevant prior art also includes U.S. patent applications: 20090112080 (Matthews, Apr. 30, 2009, “System for Measuring Electric Signals”), 20100217099 (LeBoeuf et al., Aug. 26, 2010, “Methods and Apparatus for Assessing Physiological Conditions”), 20100217100 (LeBoeuf et al., Aug. 26, 2010, “Methods and Apparatus for Measuring Physiological Conditions”), 20110098112 (LeBoeuf et al., Apr. 28, 2011, “Physiological and Environmental Monitoring Systems and Methods”), 20110106627 (LeBoeuf et al., May 5, 2011, “Physiological and Environmental Monitoring Systems and Methods”), 20120123290 (Kidmose et al., May 17, 2012, “EEG Monitoring System and Method of Monitoring an EEG”), 20120165695 (Kidmose et al., Jun. 28, 2012, “EEG Monitoring Apparatus and Method for Presenting Messages Therein”), and 20120177233 (Kidmose et al., Jul. 12, 2012, “Hearing Aid Adapted for Detecting Brain Waves and a Method for Adapting Such a Hearing Aid”).

This art further includes U.S. patent applications: 20120203081 (Leboeuf et al., Aug. 9, 2012, “Physiological and Environmental Monitoring Apparatus and Systems”), 20120209101 (Kidmose et al., Aug. 16, 2012, “Ear Plug with Surface Electrodes”), 20120235820 (Kidmose, Sep. 20, 2012, “Method and Apparatus for Alerting a Person Carrying an EEG Assembly”), 20120238856 (Kidmose et al., Sep. 20, 2012, “Portable Monitoring Device with Hearing Aid and EEG Monitor”), 20120302858 (Kidmose et al., Nov. 29, 2012, “Portable EEG Monitor System with Wireless Communication”), 20120316418 (Kilsgaard et al., Dec. 13, 2012, “Two Part EEG Monitor with Databus and Method of Communicating Between the Parts”), and 20130035578 (Chiu et al., Feb. 7, 2013, “Portable Brain Activity Monitor and Method”).

Finally, relevant prior art includes U.S. patent applications: 20130184552 (Westermann et al., Jul. 18, 2013, “Bi-Hemispheric Brain Wave System and Method of Performing Bi-Hemispherical Brain Wave Measurements”), 20130296731 (Kidmose et al., Nov. 7, 2013, “Personal EEG Monitoring Device with Electrode Validation”), 20140171775 (Kilsgaard et al., Jun. 19, 2014, “EEG Monitor with Capacitive Electrodes and a Method of Monitoring Brain Waves”), 20140316230 (Denison et al., Oct. 23, 2014, “Methods and Devices for Brain Activity Monitoring Supporting Mental State Development and Training”), 20140369537 (Pontoppidan et al., Dec. 18, 2014, “Hearing Assistance Device with Brain Computer Interface”), 20140369537 (Pontoppidan et al., Dec. 18, 2014, “Hearing Assistance Device with Brain Computer Interface”); and also WO2013026481 (Kilsgaard et al., Feb. 28, 2013, “EEG Monitor with Capacitive Electrodes and Method of Monitoring Brain Waves”).

### SUMMARY OF THE INVENTION

This invention is a wearable device for the ear comprising: an ear attachment; an electromagnetic energy sensor which is configured to collect data concerning electromagnetic energy which is transmitted through and/or emitted by the person's body; a microphone; a spectroscopic sensor which is configured to collect data concerning light energy which is transmitted through and/or reflected from the person's body; a sound-emitting unit; a power source; and a data processor. Although the prior art discloses wearable devices for the ear which include electromagnetic energy sensors, they do not appear to have the configuration disclosed in this invention which is advantageous for mobile electroencephalographic monitoring and spectroscopic analysis of body tissue

The ear attachment of this device further comprises: an inserted portion which is configured to be at least partially inserted into the ear canal; a frontal-connecting portion which is configured to span a portion of an outer ear or ear canal opening perimeter between the 11 o'clock and 4 o'clock positions, wherein clock hour positions around the perimeter of an outer ear or ear canal opening are defined relative to a 12 o'clock position that is vertically above the center of the outer opening of the ear canal; a forehead-projecting portion which is configured to span from the ear to the person's temple and/or forehead; a rear-spanning portion which is configured to span a portion of an outer ear or ear canal opening perimeter between the 5 o'clock and 1 o'clock positions, and an earlobe portion which is configured to be attached to the earlobe.

The forehead-projecting portion is configured to curve toward the center of a person's forehead as it projects forward from the person's ear. The electromagnetic energy sensor is an electroencephalographic sensor which is located on the forehead-projecting portion or the inserted portion. The spectroscopic sensor is located on the earlobe portion.

### BRIEF DESCRIPTION OF THE FIGURES

FIGS. 1 through 46 show examples of how this invention can be embodied, but they do not limit the generalizability of the claims.

FIG. 1 shows an ear-wearable device with an inserted portion, a frontal-connecting portion, a forehead-projecting portion, a rear-spanning portion, and an earlobe portion, wherein this device has an electromagnetic energy sensor, a camera, and a spectroscopic sensor.

FIG. 2 shows an ear-wearable device with an inserted portion, a frontal-connecting portion, a forehead-projecting portion, a rear-spanning portion, and an earlobe portion, wherein this device has an electromagnetic energy sensor and a camera.

FIG. 3 shows an ear-wearable device with an inserted portion, a frontal-connecting portion, a forehead-projecting portion, a rear-spanning portion, and an earlobe portion, wherein this device has an electromagnetic energy sensor and a spectroscopic sensor.

FIG. 4 shows an ear-wearable device with an inserted portion, a frontal-connecting portion, a forehead-projecting portion, a rear-spanning portion, and an earlobe portion, wherein this device has an electromagnetic energy sensor.

FIG. 5 shows an ear-wearable device with an inserted portion, a frontal-connecting portion, a rear-spanning portion, and an earlobe portion, wherein this device has an electromagnetic energy sensor, a camera, and a spectroscopic sensor.

FIG. 6 shows an ear-wearable device with an inserted portion, a frontal-connecting portion, a rear-spanning portion, and an earlobe portion, wherein this device has an electromagnetic energy sensor and a camera.

FIG. 7 shows an ear-wearable device with an inserted portion, a frontal-connecting portion, a rear-spanning portion, and an earlobe portion, wherein this device has an electromagnetic energy sensor and a spectroscopic sensor.

FIG. 8 shows an ear-wearable device with an inserted portion, a frontal-connecting portion, a rear-spanning portion, and an earlobe portion, wherein this device has an electromagnetic energy sensor.

FIG. 9 shows an ear-wearable device with an inserted portion, a frontal-connecting portion, a forehead-projecting portion, and a rear-spanning portion, wherein this device has an electromagnetic energy sensor, a camera, and a spectroscopic sensor.

FIG. 10 shows an ear-wearable device with an inserted portion, a frontal-connecting portion, a forehead-projecting portion, and a rear-spanning portion, wherein this device has an electromagnetic energy sensor and a camera.

FIG. 11 shows an ear-wearable device with an inserted portion, a frontal-connecting portion, a forehead-projecting portion, and a rear-spanning portion, wherein this device has an electromagnetic energy sensor and a spectroscopic sensor.

FIG. 12 shows an ear-wearable device with an inserted portion, a frontal-connecting portion, a forehead-projecting portion, and a rear-spanning portion, wherein this device has an electromagnetic energy sensor.

FIG. 13 shows an ear-wearable device with a frontal-connecting portion, a forehead-projecting portion, a rear-spanning portion, and an earlobe portion, wherein this device has an electromagnetic energy sensor, a camera, and a spectroscopic sensor.

FIG. 14 shows an ear-wearable device with a frontal-connecting portion, a forehead-projecting portion, a rear-spanning portion, and an earlobe portion, wherein this device has an electromagnetic energy sensor and a camera.

FIG. 15 shows an ear-wearable device with a frontal-connecting portion, a forehead-projecting portion, a rear-spanning portion, and an earlobe portion, wherein this device has an electromagnetic energy sensor and a spectroscopic sensor.

FIG. 16 shows an ear-wearable device with a frontal-connecting portion, a forehead-projecting portion, a rear-spanning portion, and an earlobe portion, wherein this device has an electromagnetic energy sensor.

FIG. 17 shows an ear-wearable device with an inserted portion, a frontal-connecting portion, and a rear-spanning portion, wherein this device has an electromagnetic energy sensor, a camera, and a spectroscopic sensor.

FIG. 18 shows an ear-wearable device with an inserted portion, a frontal-connecting portion, and a rear-spanning portion, wherein this device has an electromagnetic energy sensor and a camera.

FIG. 19 shows an ear-wearable device with an inserted portion, a frontal-connecting portion, and a rear-spanning portion, wherein this device has an electromagnetic energy sensor and a spectroscopic sensor.

FIG. 20 shows an ear-wearable device with an inserted portion, a frontal-connecting portion, and a rear-spanning portion, wherein this device has an electromagnetic energy sensor.

FIG. 21 shows an ear-wearable device with a frontal-connecting portion, a forehead-projecting portion, and a rear-spanning portion, wherein this device has an electromagnetic energy sensor, a camera, and a spectroscopic sensor.

FIG. 22 shows an ear-wearable device with a frontal-connecting portion, a forehead-projecting portion, and a rear-spanning portion, wherein this device has an electromagnetic energy sensor and a camera.

FIG. 23 shows an ear-wearable device with a frontal-connecting portion, a forehead-projecting portion, and a rear-spanning portion, wherein this device has an electromagnetic energy sensor and a spectroscopic sensor.

FIG. 24 shows an ear-wearable device with a frontal-connecting portion, a forehead-projecting portion, and a rear-spanning portion, wherein this device has an electromagnetic energy sensor.

FIG. 25 shows an ear-wearable device with a frontal-connecting portion, a rear-spanning portion, and an earlobe portion, wherein this device has an electromagnetic energy sensor, a camera, and a spectroscopic sensor.

FIG. 26 shows an ear-wearable device with a frontal-connecting portion, a rear-spanning portion, and an earlobe portion, wherein this device has an electromagnetic energy sensor and a camera.

FIG. 27 shows an ear-wearable device with a frontal-connecting portion, a rear-spanning portion, and an earlobe portion, wherein this device has an electromagnetic energy sensor and a spectroscopic sensor.

FIG. 28 shows an ear-wearable device with a frontal-connecting portion, a rear-spanning portion, and an earlobe portion, wherein this device has an electromagnetic energy sensor.

FIG. 29 shows an ear-wearable device with a forehead-projecting portion, a rear-spanning portion, and an earlobe portion, wherein this device has an electromagnetic energy sensor, a camera, and a spectroscopic sensor.

FIG. 30 shows an ear-wearable device with a forehead-projecting portion, a rear-spanning portion, and an earlobe portion, wherein this device has an electromagnetic energy sensor and a camera.

FIG. 31 shows an ear-wearable device with a forehead-projecting portion, a rear-spanning portion, and an earlobe portion, wherein this device has an electromagnetic energy sensor and a spectroscopic sensor.

FIG. 32 shows an ear-wearable device with a forehead-projecting portion, a rear-spanning portion, and an earlobe portion, wherein this device has an electromagnetic energy sensor.

FIG. 33 shows an ear-wearable device with a frontal-connecting portion and a rear-spanning portion, wherein this device has an electromagnetic energy sensor, a camera, and a spectroscopic sensor.

FIG. 34 shows an ear-wearable device with a frontal-connecting portion and a rear-spanning portion, wherein this device has an electromagnetic energy sensor and a camera.

FIG. 35 shows an ear-wearable device with a frontal-connecting portion and a rear-spanning portion, wherein this device has an electromagnetic energy sensor and a spectroscopic sensor.

FIG. 36 shows an ear-wearable device with a frontal-connecting portion and a rear-spanning portion, wherein this device has an electromagnetic energy sensor.

FIG. 37 shows an ear-wearable device with an inserted portion and an earlobe portion, wherein this device has an electromagnetic energy sensor, a camera, and a spectroscopic sensor.

FIG. 38 shows an ear-wearable device with an inserted portion and an earlobe portion, wherein this device has an electromagnetic energy sensor and a camera.

FIG. 39 shows an ear-wearable device with an inserted portion and an earlobe portion, wherein this device has an electromagnetic energy sensor and a spectroscopic sensor.

FIG. 40 shows an ear-wearable device with an inserted portion and an earlobe portion, wherein this device has an electromagnetic energy sensor.

FIG. 41 shows an earlobe attachment with an electromagnetic energy sensor, a camera, and a spectroscopic sensor.

FIG. 42 shows an earlobe attachment with an electromagnetic energy sensor and a camera.

FIG. 43 shows an earlobe attachment with an electromagnetic energy sensor and a spectroscopic sensor.

FIG. 44 shows an earlobe attachment with an electromagnetic energy sensor.

FIG. 45 shows an ear insert with an electromagnetic energy sensor and a spectroscopic sensor.

FIG. 46 shows an ear insert with an electromagnetic energy sensor.

FIG. 47 shows how radial clock hour (or degree) positions can be defined for the perimeter of an outer ear or ear canal outer opening.

### DETAILED DESCRIPTION OF THE FIGURES

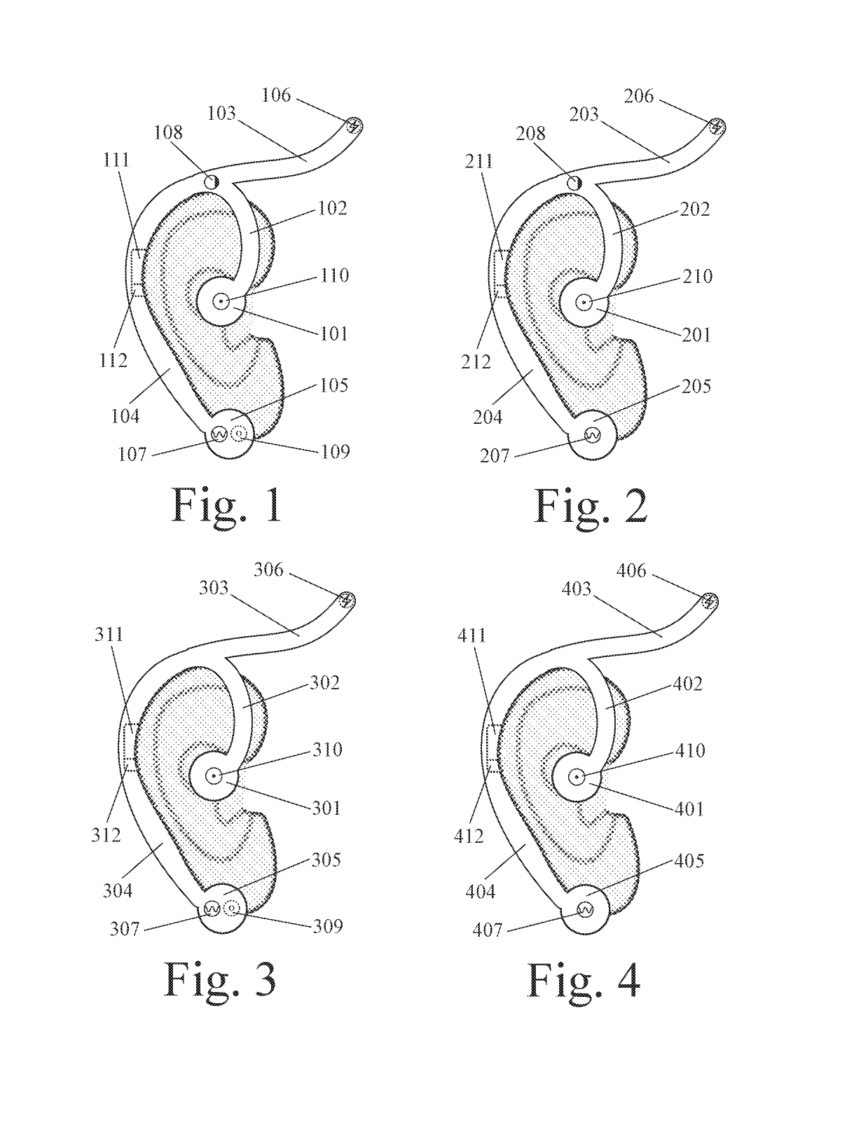

FIGS. 1 through 46 show several examples of how this invention can be embodied, but they do not limit the full generalizability of the claims. FIG. 1 shows an example of how this invention can be embodied in a wearable device for the ear comprising: an ear attachment, wherein this ear attachment further comprises an inserted portion 101 which is configured to be at least partially inserted into the ear canal, a frontal-connecting portion 102 which is configured to span a portion of an outer ear or ear canal opening perimeter between 11 o'clock (or 330 degree) and 4 o'clock (or 120 degree) positions, a forehead-projecting portion 103 which is configured to span from the ear to the person's temple and/or forehead, a rear-spanning portion 104 which is configured to span a portion of an outer ear or ear canal opening perimeter between 5 o'clock (or 150 degree) and 1 o'clock (or 30 degree) positions, and an earlobe portion 105 which is configured to be attached to the earlobe; an electromagnetic energy sensor 106 which is configured to collect data concerning electromagnetic energy which is transmitted through and/or emitted by the person's body, which is located on the forehead-projecting portion; a microphone 107, which is located on the earlobe portion; a camera 108, which is located on the frontal-connecting portion; a spectroscopic sensor 109 which is configured to collect data concerning light energy which is transmitted through and/or reflected from the person's body, which is located on the earlobe portion; a sound-emitting unit 110, which is located on the inserted portion; a power source 111; and a data processor 112.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; inserted portion; earlobe portion; and rear-spanning portion.

In an example, a microphone can be located on a portion of an ear attachment selected from the group consisting of: earlobe portion; frontal-connecting portion; forehead-projecting portion; inserted portion; and rear-spanning portion. In an example, a camera can be located on a portion of an ear attachment selected from the group consisting of: frontal-connecting portion; forehead-projecting portion; and earlobe portion. In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion; inserted portion; and rear-spanning portion.

In an example, a sound-emitting unit can be a speaker. In an example, a sound-emitting unit can be located on a portion of an ear attachment selected from the group consisting of: inserted portion; frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 2 shows an example of how this invention can be embodied in a wearable device for the ear comprising: an ear attachment, wherein this ear attachment further comprises an inserted portion 201 which is configured to be at least partially inserted into the ear canal, a frontal-connecting portion 202 which is configured to span a portion of an outer ear or ear canal opening perimeter between 11 o'clock (or 330 degree) and 4 o'clock (or 120 degree) positions, a forehead-projecting portion 203 which is configured to span from the ear to the person's temple and/or forehead, a rear-spanning portion 204 which is configured to span a portion of an outer ear or ear canal opening perimeter between 5 o'clock (or 150 degree) and 1 o'clock (or 30 degree) positions, and an earlobe portion 205 which is configured to be attached to the earlobe; an electromagnetic energy sensor 206 which is configured to collect data concerning electromagnetic energy which is transmitted through and/or emitted by the person's body, which is located on the forehead-projecting portion; a microphone 207, which is located on the earlobe portion; a camera 208, which is located on the frontal-connecting portion; a sound-emitting unit 210, which is located on the inserted portion; a power source 211; and a data processor 212.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; inserted portion; earlobe portion; and rear-spanning portion.

In an example, a microphone can be located on a portion of an ear attachment selected from the group consisting of: earlobe portion; frontal-connecting portion; forehead-projecting portion; inserted portion; and rear-spanning portion. In an example, a camera can be located on a portion of an ear attachment selected from the group consisting of: frontal-connecting portion; forehead-projecting portion; and earlobe portion. In an example, a sound-emitting unit can be a speaker. In an example, a sound-emitting unit can be located on a portion of an ear attachment selected from the group consisting of: inserted portion; frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 3 shows an example of how this invention can be embodied in a wearable device for the ear comprising: an ear attachment, wherein this ear attachment further comprises an inserted portion 301 which is configured to be at least partially inserted into the ear canal, a frontal-connecting portion 302 which is configured to span a portion of an outer ear or ear canal opening perimeter between 11 o'clock (or 330 degree) and 4 o'clock (or 120 degree) positions, a forehead-projecting portion 303 which is configured to span from the ear to the person's temple and/or forehead, a rear-spanning portion 304 which is configured to span a portion of an outer ear or ear canal opening perimeter between 5 o'clock (or 150 degree) and 1 o'clock (or 30 degree) positions, and an earlobe portion 305 which is configured to be attached to the earlobe; an electromagnetic energy sensor 306 which is configured to collect data concerning electromagnetic energy which is transmitted through and/or emitted by the person's body, which is located on the forehead-projecting portion; a microphone 307, which is located on the earlobe portion; a spectroscopic sensor 309 which is configured to collect data concerning light energy which is transmitted through and/or reflected from the person's body, which is located on the earlobe portion; a sound-emitting unit 310, which is located on the inserted portion; a power source 311; and a data processor 312.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; inserted portion; earlobe portion; and rear-spanning portion.

In an example, a microphone can be located on a portion of an ear attachment selected from the group consisting of: earlobe portion; frontal-connecting portion; forehead-projecting portion; inserted portion; and rear-spanning portion. In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion; inserted portion; and rear-spanning portion.

In an example, a sound-emitting unit can be a speaker. In an example, a sound-emitting unit can be located on a portion of an ear attachment selected from the group consisting of: inserted portion; frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 4 shows an example of how this invention can be embodied in a wearable device for the ear comprising: an ear attachment, wherein this ear attachment further comprises an inserted portion 401 which is configured to be at least partially inserted into the ear canal, a frontal-connecting portion 402 which is configured to span a portion of an outer ear or ear canal opening perimeter between 11 o'clock (or 330 degree) and 4 o'clock (or 120 degree) positions, a forehead-projecting portion 403 which is configured to span from the ear to the person's temple and/or forehead, a rear-spanning portion 404 which is configured to span a portion of an outer ear or ear canal opening perimeter between 5 o'clock (or 150 degree) and 1 o'clock (or 30 degree) positions, and an earlobe portion 405 which is configured to be attached to the earlobe; an electromagnetic energy sensor 406 which is configured to collect data concerning electromagnetic energy which is transmitted through and/or emitted by the person's body, which is located on the forehead-projecting portion; a microphone 407, which is located on the earlobe portion; a sound-emitting unit 410, which is located on the inserted portion; a power source 411; and a data processor 412.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; inserted portion; earlobe portion; and rear-spanning portion.

In an example, a microphone can be located on a portion of an ear attachment selected from the group consisting of: earlobe portion; frontal-connecting portion; forehead-projecting portion; inserted portion; and rear-spanning portion. In an example, a sound-emitting unit can be a speaker. In an example, a sound-emitting unit can be located on a portion of an ear attachment selected from the group consisting of: inserted portion; frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

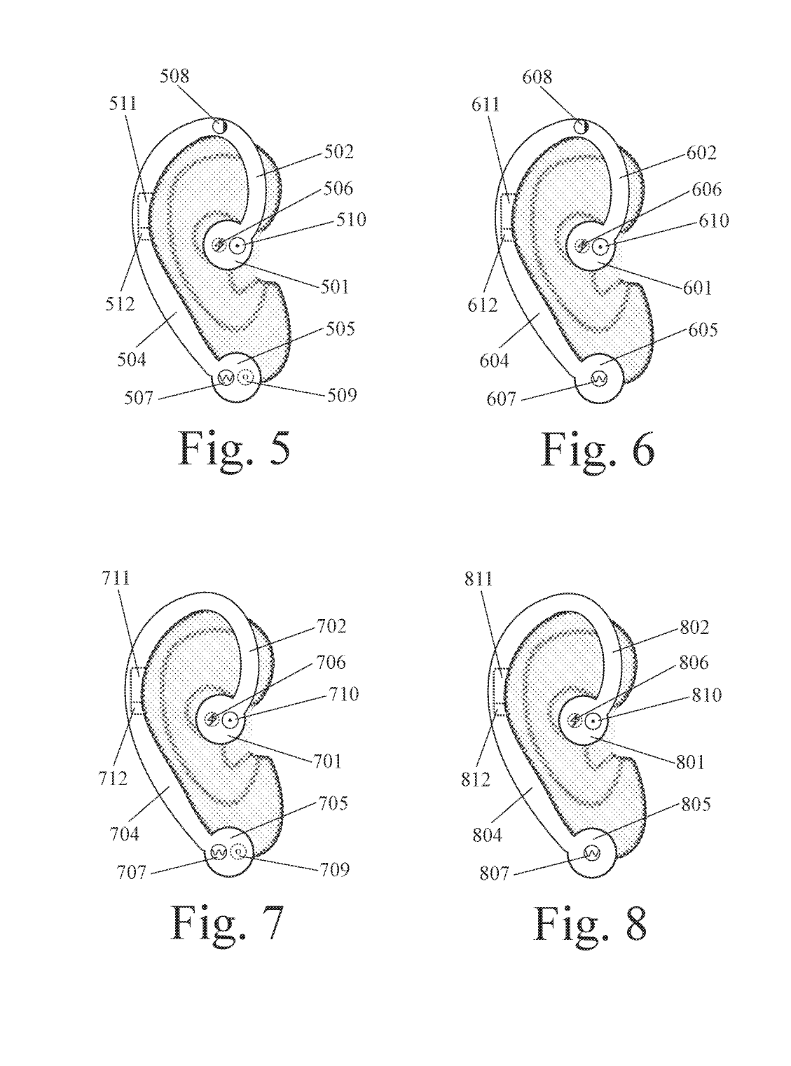

FIG. 5 shows an example of how this invention can be embodied in a wearable device for the ear comprising: an ear attachment, wherein this ear attachment further comprises an inserted portion 501 which is configured to be at least partially inserted into the ear canal, a frontal-connecting portion 502 which is configured to span a portion of an outer ear or ear canal opening perimeter between 11 o'clock (or 330 degree) and 4 o'clock (or 120 degree) positions, a rear-spanning portion 504 which is configured to span a portion of an outer ear or ear canal opening perimeter between 5 o'clock (or 150 degree) and 1 o'clock (or 30 degree) positions, and an earlobe portion 505 which is configured to be attached to the earlobe; an electromagnetic energy sensor 506 which is configured to collect data concerning electromagnetic energy which is transmitted through and/or emitted by the person's body, which is located on the inserted portion; a microphone 507, which is located on the earlobe portion; a camera 508, which is located on the frontal-connecting portion; a spectroscopic sensor 509 which is configured to collect data concerning light energy which is transmitted through and/or reflected from the person's body, which is located on the earlobe portion; a sound-emitting unit 510, which is located on the inserted portion; a power source 511; and a data processor 512.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion; earlobe portion; and rear-spanning portion. In an example, a microphone can be located on a portion of an ear attachment selected from the group consisting of: earlobe portion; frontal-connecting portion; inserted portion; and rear-spanning portion. In an example, a camera can be located on a portion of an ear attachment selected from the group consisting of: frontal-connecting portion; and earlobe portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion; inserted portion; and rear-spanning portion.

In an example, a sound-emitting unit can be a speaker. In an example, a sound-emitting unit can be located on a portion of an ear attachment selected from the group consisting of: inserted portion; frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 6 shows an example of how this invention can be embodied in a wearable device for the ear comprising: an ear attachment, wherein this ear attachment further comprises an inserted portion 601 which is configured to be at least partially inserted into the ear canal, a frontal-connecting portion 602 which is configured to span a portion of an outer ear or ear canal opening perimeter between 11 o'clock (or 330 degree) and 4 o'clock (or 120 degree) positions, a rear-spanning portion 604 which is configured to span a portion of an outer ear or ear canal opening perimeter between 5 o'clock (or 150 degree) and 1 o'clock (or 30 degree) positions, and an earlobe portion 605 which is configured to be attached to the earlobe; an electromagnetic energy sensor 606 which is configured to collect data concerning electromagnetic energy which is transmitted through and/or emitted by the person's body, which is located on the inserted portion; a microphone 607, which is located on the earlobe portion; a camera 608, which is located on the frontal-connecting portion; a sound-emitting unit 610, which is located on the inserted portion; a power source 611; and a data processor 612.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion; earlobe portion; and rear-spanning portion.

In an example, a microphone can be located on a portion of an ear attachment selected from the group consisting of: earlobe portion; frontal-connecting portion; inserted portion; and rear-spanning portion. In an example, a camera can be located on a portion of an ear attachment selected from the group consisting of: frontal-connecting portion; and earlobe portion. In an example, a sound-emitting unit can be a speaker. In an example, a sound-emitting unit can be located on a portion of an ear attachment selected from the group consisting of: inserted portion; frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 7 shows an example of how this invention can be embodied in a wearable device for the ear comprising: an ear attachment, wherein this ear attachment further comprises an inserted portion 701 which is configured to be at least partially inserted into the ear canal, a frontal-connecting portion 702 which is configured to span a portion of an outer ear or ear canal opening perimeter between 11 o'clock (or 330 degree) and 4 o'clock (or 120 degree) positions, a rear-spanning portion 704 which is configured to span a portion of an outer ear or ear canal opening perimeter between 5 o'clock (or 150 degree) and 1 o'clock (or 30 degree) positions, and an earlobe portion 705 which is configured to be attached to the earlobe; an electromagnetic energy sensor 706 which is configured to collect data concerning electromagnetic energy which is transmitted through and/or emitted by the person's body, which is located on the inserted portion; a microphone 707, which is located on the earlobe portion; a spectroscopic sensor 709 which is configured to collect data concerning light energy which is transmitted through and/or reflected from the person's body, which is located on the earlobe portion; a sound-emitting unit 710, which is located on the inserted portion; a power source 711; and a data processor 712.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion; earlobe portion; and rear-spanning portion. In an example, a microphone can be located on a portion of an ear attachment selected from the group consisting of: earlobe portion; frontal-connecting portion; inserted portion; and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion; inserted portion; and rear-spanning portion.

In an example, a sound-emitting unit can be a speaker. In an example, a sound-emitting unit can be located on a portion of an ear attachment selected from the group consisting of: inserted portion; frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 8 shows an example of how this invention can be embodied in a wearable device for the ear comprising: an ear attachment, wherein this ear attachment further comprises an inserted portion 801 which is configured to be at least partially inserted into the ear canal, a frontal-connecting portion 802 which is configured to span a portion of an outer ear or ear canal opening perimeter between 11 o'clock (or 330 degree) and 4 o'clock (or 120 degree) positions, a rear-spanning portion 804 which is configured to span a portion of an outer ear or ear canal opening perimeter between 5 o'clock (or 150 degree) and 1 o'clock (or 30 degree) positions, and an earlobe portion 805 which is configured to be attached to the earlobe; an electromagnetic energy sensor 806 which is configured to collect data concerning electromagnetic energy which is transmitted through and/or emitted by the person's body, which is located on the inserted portion; a microphone 807, which is located on the earlobe portion; a sound-emitting unit 810, which is located on the inserted portion; a power source 811; and a data processor 812.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion; earlobe portion; and rear-spanning portion.

In an example, a microphone can be located on a portion of an ear attachment selected from the group consisting of: earlobe portion; frontal-connecting portion; inserted portion; and rear-spanning portion. In an example, a sound-emitting unit can be a speaker. In an example, a sound-emitting unit can be located on a portion of an ear attachment selected from the group consisting of: inserted portion; frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

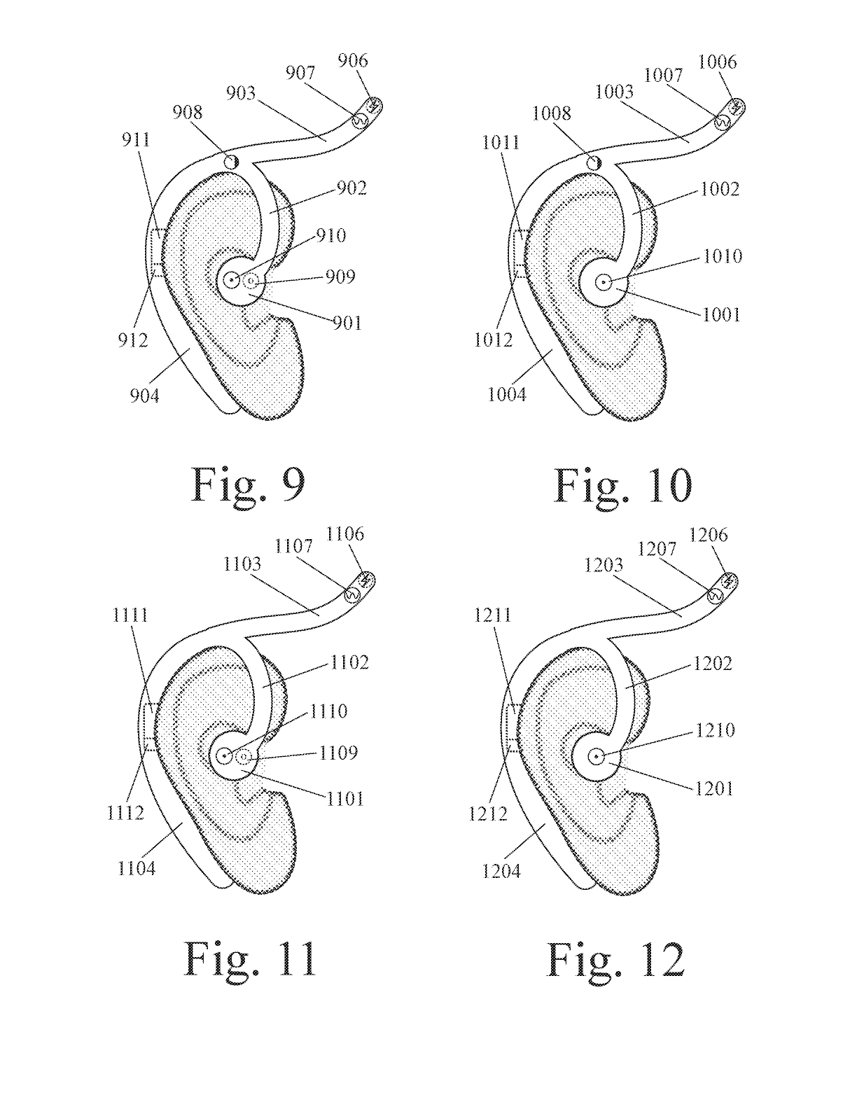

FIG. 9 shows an example of how this invention can be embodied in a wearable device for the ear comprising: an ear attachment, wherein this ear attachment further comprises an inserted portion 901 which is configured to be at least partially inserted into the ear canal, a frontal-connecting portion 902 which is configured to span a portion of an outer ear or ear canal opening perimeter between 11 o'clock (or 330 degree) and 4 o'clock (or 120 degree) positions, a forehead-projecting portion 903 which is configured to span from the ear to the person's temple and/or forehead, a rear-spanning portion 904 which is configured to span a portion of an outer ear or ear canal opening perimeter between 5 o'clock (or 150 degree) and 1 o'clock (or 30 degree) positions; an electromagnetic energy sensor 906 which is configured to collect data concerning electromagnetic energy which is transmitted through and/or emitted by the person's body, which is located on the forehead-projecting portion; a microphone 907, which is located on the forehead-projecting portion; a camera 908, which is located on the frontal-connecting portion; a spectroscopic sensor 909 which is configured to collect data concerning light energy which is transmitted through and/or reflected from the person's body, which is located on the inserted portion; a sound-emitting unit 910, which is located on the inserted portion; a power source 911; and a data processor 912.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; inserted portion; and rear-spanning portion. In an example, a microphone can be located on a portion of an ear attachment selected from the group consisting of: frontal-connecting portion; forehead-projecting portion; inserted portion; and rear-spanning portion. In an example, a camera can be located on a portion of an ear attachment selected from the group consisting of: frontal-connecting portion; and forehead-projecting portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: inserted portion; and rear-spanning portion.

In an example, a sound-emitting unit can be a speaker. In an example, a sound-emitting unit can be located on a portion of an ear attachment selected from the group consisting of: inserted portion; frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 10 shows an example of how this invention can be embodied in a wearable device for the ear comprising: an ear attachment, wherein this ear attachment further comprises an inserted portion 1001 which is configured to be at least partially inserted into the ear canal, a frontal-connecting portion 1002 which is configured to span a portion of an outer ear or ear canal opening perimeter between 11 o'clock (or 330 degree) and 4 o'clock (or 120 degree) positions, a forehead-projecting portion 1003 which is configured to span from the ear to the person's temple and/or forehead, a rear-spanning portion 1004 which is configured to span a portion of an outer ear or ear canal opening perimeter between 5 o'clock (or 150 degree) and 1 o'clock (or 30 degree) positions; an electromagnetic energy sensor 1006 which is configured to collect data concerning electromagnetic energy which is transmitted through and/or emitted by the person's body, which is located on the forehead-projecting portion; a microphone 1007, which is located on the forehead-projecting portion; a camera 1008, which is located on the frontal-connecting portion; a sound-emitting unit 1010, which is located on the inserted portion; a power source 1011; and a data processor 1012.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; inserted portion; and rear-spanning portion.

In an example, a microphone can be located on a portion of an ear attachment selected from the group consisting of: frontal-connecting portion; forehead-projecting portion; inserted portion; and rear-spanning portion. In an example, a camera can be located on a portion of an ear attachment selected from the group consisting of: frontal-connecting portion; and forehead-projecting portion. In an example, a sound-emitting unit can be a speaker. In an example, a sound-emitting unit can be located on a portion of an ear attachment selected from the group consisting of: inserted portion; frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 11 shows an example of how this invention can be embodied in a wearable device for the ear comprising: an ear attachment, wherein this ear attachment further comprises an inserted portion 1101 which is configured to be at least partially inserted into the ear canal, a frontal-connecting portion 1102 which is configured to span a portion of an outer ear or ear canal opening perimeter between 11 o'clock (or 330 degree) and 4 o'clock (or 120 degree) positions, a forehead-projecting portion 1103 which is configured to span from the ear to the person's temple and/or forehead, a rear-spanning portion 1104 which is configured to span a portion of an outer ear or ear canal opening perimeter between 5 o'clock (or 150 degree) and 1 o'clock (or 30 degree) positions; an electromagnetic energy sensor 1106 which is configured to collect data concerning electromagnetic energy which is transmitted through and/or emitted by the person's body, which is located on the forehead-projecting portion; a microphone 1107, which is located on the forehead-projecting portion; a spectroscopic sensor 1109 which is configured to collect data concerning light energy which is transmitted through and/or reflected from the person's body, which is located on the inserted portion; a sound-emitting unit 1110, which is located on the inserted portion; a power source 1111; and a data processor 1112.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; inserted portion; and rear-spanning portion. In an example, a microphone can be located on a portion of an ear attachment selected from the group consisting of: frontal-connecting portion; forehead-projecting portion; inserted portion; and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: inserted portion; and rear-spanning portion.

In an example, a sound-emitting unit can be a speaker. In an example, a sound-emitting unit can be located on a portion of an ear attachment selected from the group consisting of: inserted portion; frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 12 shows an example of how this invention can be embodied in a wearable device for the ear comprising: an ear attachment, wherein this ear attachment further comprises an inserted portion 1201 which is configured to be at least partially inserted into the ear canal, a frontal-connecting portion 1202 which is configured to span a portion of an outer ear or ear canal opening perimeter between 11 o'clock (or 330 degree) and 4 o'clock (or 120 degree) positions, a forehead-projecting portion 1203 which is configured to span from the ear to the person's temple and/or forehead, a rear-spanning portion 1204 which is configured to span a portion of an outer ear or ear canal opening perimeter between 5 o'clock (or 150 degree) and 1 o'clock (or 30 degree) positions; an electromagnetic energy sensor 1206 which is configured to collect data concerning electromagnetic energy which is transmitted through and/or emitted by the person's body, which is located on the forehead-projecting portion; a microphone 1207, which is located on the forehead-projecting portion; a sound-emitting unit 1210, which is located on the inserted portion; a power source 1211; and a data processor 1212.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; inserted portion; and rear-spanning portion.

In an example, a microphone can be located on a portion of an ear attachment selected from the group consisting of: frontal-connecting portion; forehead-projecting portion; inserted portion; and rear-spanning portion. In an example, a sound-emitting unit can be a speaker. In an example, a sound-emitting unit can be located on a portion of an ear attachment selected from the group consisting of: inserted portion; frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 13 shows an example of how this invention can be embodied in a wearable device for the ear comprising: an ear attachment, wherein this ear attachment further comprises a frontal-connecting portion 1302 which is configured to span a portion of an outer ear or ear canal opening perimeter between 11 o'clock (or 330 degree) and 4 o'clock (or 120 degree) positions, a forehead-projecting portion 1303 which is configured to span from the ear to the person's temple and/or forehead, a rear-spanning portion 1304 which is configured to span a portion of an outer ear or ear canal opening perimeter between 5 o'clock (or 150 degree) and 1 o'clock (or 30 degree) positions, and an earlobe portion 1305 which is configured to be attached to the earlobe; an electromagnetic energy sensor 1306 which is configured to collect data concerning electromagnetic energy which is transmitted through and/or emitted by the person's body, which is located on the forehead-projecting portion; a microphone 1307, which is located on the earlobe portion; a camera 1308, which is located on the frontal-connecting portion; a spectroscopic sensor 1309 which is configured to collect data concerning light energy which is transmitted through and/or reflected from the person's body, which is located on the earlobe portion; a sound-emitting unit 1310, which is located on the frontal-connecting portion; a power source 1311; and a data processor 1312.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; earlobe portion; and rear-spanning portion.

In an example, a microphone can be located on a portion of an ear attachment selected from the group consisting of: earlobe portion; frontal-connecting portion; forehead-projecting portion; and rear-spanning portion. In an example, a camera can be located on a portion of an ear attachment selected from the group consisting of: frontal-connecting portion; forehead-projecting portion; and earlobe portion. In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion; and rear-spanning portion.

In an example, a sound-emitting unit can be a speaker. In an example, a sound-emitting unit can be located on a portion of an ear attachment selected from the group consisting of: frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

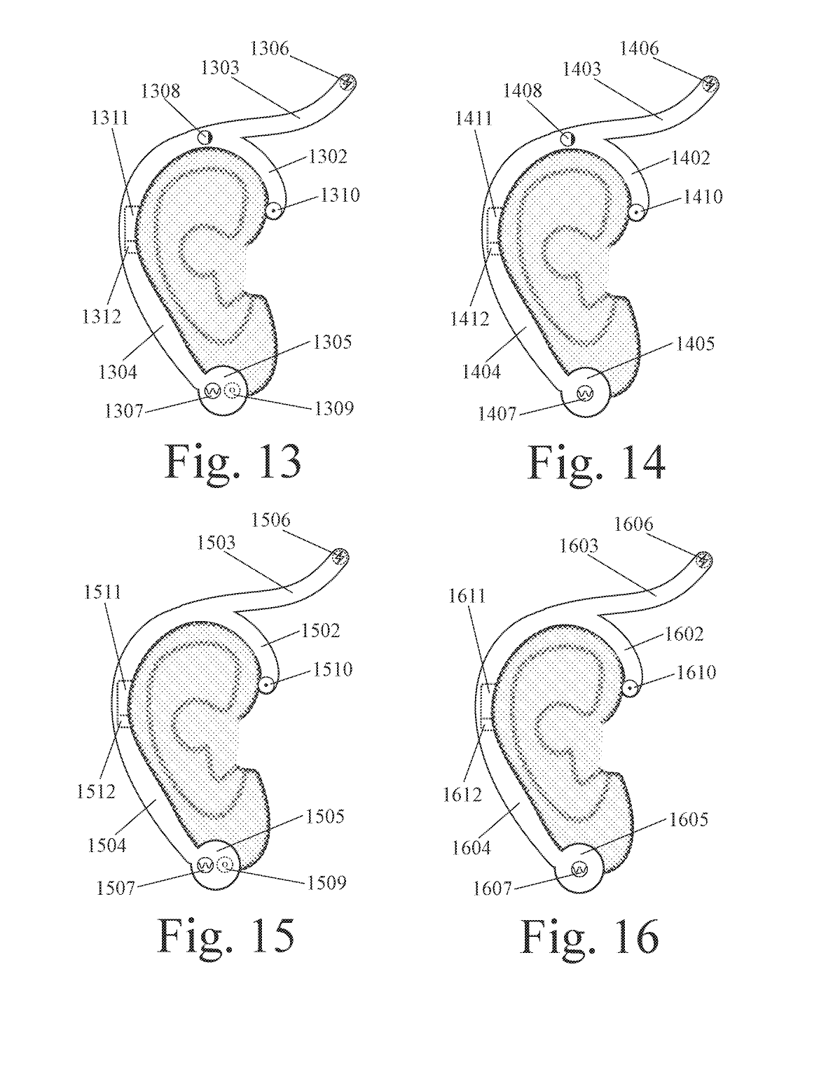

FIG. 14 shows an example of how this invention can be embodied in a wearable device for the ear comprising: an ear attachment, wherein this ear attachment further comprises a frontal-connecting portion 1402 which is configured to span a portion of an outer ear or ear canal opening perimeter between 11 o'clock (or 330 degree) and 4 o'clock (or 120 degree) positions, a forehead-projecting portion 1403 which is configured to span from the ear to the person's temple and/or forehead, a rear-spanning portion 1404 which is configured to span a portion of an outer ear or ear canal opening perimeter between 5 o'clock (or 150 degree) and 1 o'clock (or 30 degree) positions, and an earlobe portion 1405 which is configured to be attached to the earlobe; an electromagnetic energy sensor 1406 which is configured to collect data concerning electromagnetic energy which is transmitted through and/or emitted by the person's body, which is located on the forehead-projecting portion; a microphone 1407, which is located on the earlobe portion; a camera 1408, which is located on the frontal-connecting portion; a sound-emitting unit 1410, which is located on the frontal-connecting portion; a power source 1411; and a data processor 1412.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; earlobe portion; and rear-spanning portion.

In an example, a microphone can be located on a portion of an ear attachment selected from the group consisting of: earlobe portion; frontal-connecting portion; forehead-projecting portion; and rear-spanning portion. In an example, a camera can be located on a portion of an ear attachment selected from the group consisting of: frontal-connecting portion; forehead-projecting portion; and earlobe portion. In an example, a sound-emitting unit can be a speaker. In an example, a sound-emitting unit can be located on a portion of an ear attachment selected from the group consisting of: frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 15 shows an example of how this invention can be embodied in a wearable device for the ear comprising: an ear attachment, wherein this ear attachment further comprises a frontal-connecting portion 1502 which is configured to span a portion of an outer ear or ear canal opening perimeter between 11 o'clock (or 330 degree) and 4 o'clock (or 120 degree) positions, a forehead-projecting portion 1503 which is configured to span from the ear to the person's temple and/or forehead, a rear-spanning portion 1504 which is configured to span a portion of an outer ear or ear canal opening perimeter between 5 o'clock (or 150 degree) and 1 o'clock (or 30 degree) positions, and an earlobe portion 1505 which is configured to be attached to the earlobe; an electromagnetic energy sensor 1506 which is configured to collect data concerning electromagnetic energy which is transmitted through and/or emitted by the person's body, which is located on the forehead-projecting portion; a microphone 1507, which is located on the earlobe portion; a spectroscopic sensor 1509 which is configured to collect data concerning light energy which is transmitted through and/or reflected from the person's body, which is located on the earlobe portion; a sound-emitting unit 1510, which is located on the frontal-connecting portion; a power source 1511; and a data processor 1512.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; earlobe portion; and rear-spanning portion. In an example, a microphone can be located on a portion of an ear attachment selected from the group consisting of: earlobe portion; frontal-connecting portion; forehead-projecting portion; and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion; and rear-spanning portion.

In an example, a sound-emitting unit can be a speaker. In an example, a sound-emitting unit can be located on a portion of an ear attachment selected from the group consisting of: frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 16 shows an example of how this invention can be embodied in a wearable device for the ear comprising: an ear attachment, wherein this ear attachment further comprises a frontal-connecting portion 1602 which is configured to span a portion of an outer ear or ear canal opening perimeter between 11 o'clock (or 330 degree) and 4 o'clock (or 120 degree) positions, a forehead-projecting portion 1603 which is configured to span from the ear to the person's temple and/or forehead, a rear-spanning portion 1604 which is configured to span a portion of an outer ear or ear canal opening perimeter between 5 o'clock (or 150 degree) and 1 o'clock (or 30 degree) positions, and an earlobe portion 1605 which is configured to be attached to the earlobe; an electromagnetic energy sensor 1606 which is configured to collect data concerning electromagnetic energy which is transmitted through and/or emitted by the person's body, which is located on the forehead-projecting portion; a microphone 1607, which is located on the earlobe portion; a sound-emitting unit 1610, which is located on the frontal-connecting portion; a power source 1611; and a data processor 1612.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; earlobe portion; and rear-spanning portion.

In an example, a microphone can be located on a portion of an ear attachment selected from the group consisting of: earlobe portion; frontal-connecting portion; forehead-projecting portion; and rear-spanning portion. In an example, a sound-emitting unit can be a speaker. In an example, a sound-emitting unit can be located on a portion of an ear attachment selected from the group consisting of: frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

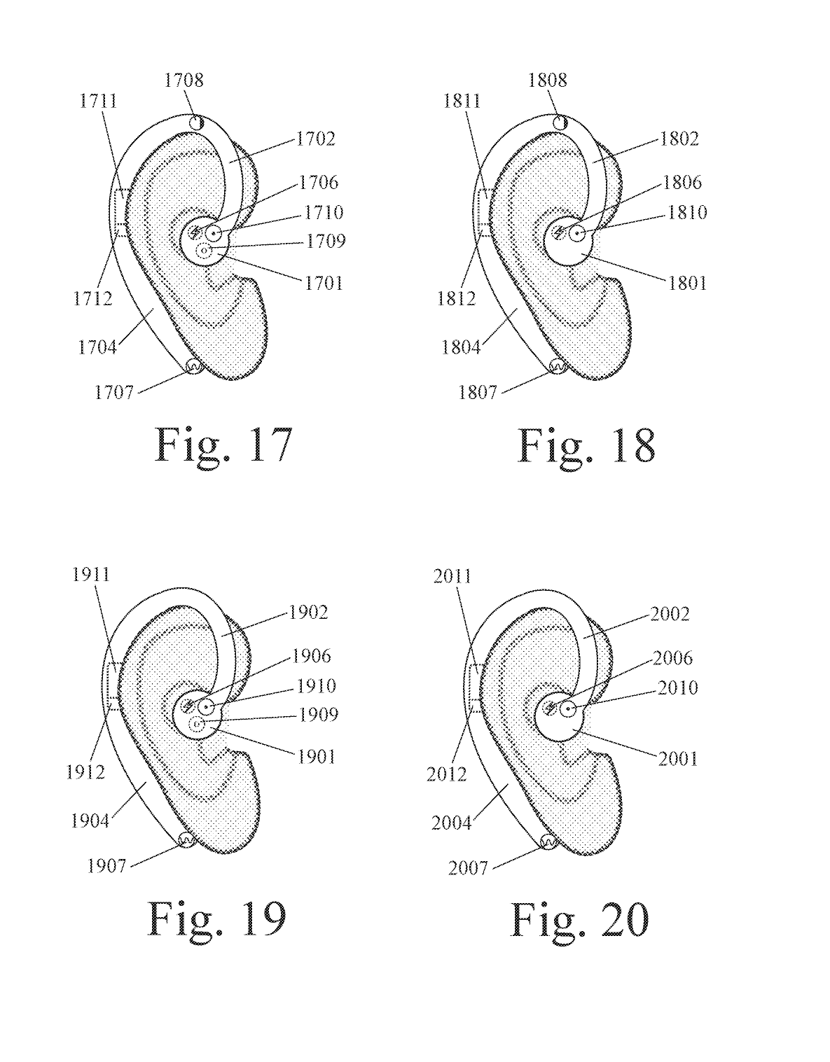

FIG. 17 shows an example of how this invention can be embodied in a wearable device for the ear comprising: an ear attachment, wherein this ear attachment further comprises an inserted portion 1701 which is configured to be at least partially inserted into the ear canal, a frontal-connecting portion 1702 which is configured to span a portion of an outer ear or ear canal opening perimeter between 11 o'clock (or 330 degree) and 4 o'clock (or 120 degree) positions, a rear-spanning portion 1704 which is configured to span a portion of an outer ear or ear canal opening perimeter between 5 o'clock (or 150 degree) and 1 o'clock (or 30 degree) positions; an electromagnetic energy sensor 1706 which is configured to collect data concerning electromagnetic energy which is transmitted through and/or emitted by the person's body, which is located on the inserted portion; a microphone 1707, which is located on the rear-spanning portion; a camera 1708, which is located on the frontal-connecting portion; a spectroscopic sensor 1709 which is configured to collect data concerning light energy which is transmitted through and/or reflected from the person's body, which is located on the inserted portion; a sound-emitting unit 1710, which is located on the inserted portion; a power source 1711; and a data processor 1712.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion and rear-spanning portion. In an example, a microphone can be located on a portion of an ear attachment selected from the group consisting of: frontal-connecting portion; inserted portion; and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: inserted portion and rear-spanning portion.

In an example, a sound-emitting unit can be a speaker. In an example, a sound-emitting unit can be located on a portion of an ear attachment selected from the group consisting of: inserted portion; frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 18 shows an example of how this invention can be embodied in a wearable device for the ear comprising: an ear attachment, wherein this ear attachment further comprises an inserted portion 1801 which is configured to be at least partially inserted into the ear canal, a frontal-connecting portion 1802 which is configured to span a portion of an outer ear or ear canal opening perimeter between 11 o'clock (or 330 degree) and 4 o'clock (or 120 degree) positions, a rear-spanning portion 1804 which is configured to span a portion of an outer ear or ear canal opening perimeter between 5 o'clock (or 150 degree) and 1 o'clock (or 30 degree) positions; an electromagnetic energy sensor 1806 which is configured to collect data concerning electromagnetic energy which is transmitted through and/or emitted by the person's body, which is located on the inserted portion; a microphone 1807, which is located on the rear-spanning portion; a camera 1808, which is located on the frontal-connecting portion; a sound-emitting unit 1810, which is located on the inserted portion; a power source 1811; and a data processor 1812.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion; and rear-spanning portion.

In an example, a microphone can be located on a portion of an ear attachment selected from the group consisting of: frontal-connecting portion; inserted portion; and rear-spanning portion. In an example, a sound-emitting unit can be a speaker. In an example, a sound-emitting unit can be located on a portion of an ear attachment selected from the group consisting of: inserted portion; frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 19 shows an example of how this invention can be embodied in a wearable device for the ear comprising: an ear attachment, wherein this ear attachment further comprises an inserted portion 1901 which is configured to be at least partially inserted into the ear canal, a frontal-connecting portion 1902 which is configured to span a portion of an outer ear or ear canal opening perimeter between 11 o'clock (or 330 degree) and 4 o'clock (or 120 degree) positions, a rear-spanning portion 1904 which is configured to span a portion of an outer ear or ear canal opening perimeter between 5 o'clock (or 150 degree) and 1 o'clock (or 30 degree) positions; an electromagnetic energy sensor 1906 which is configured to collect data concerning electromagnetic energy which is transmitted through and/or emitted by the person's body, which is located on the inserted portion; a microphone 1907, which is located on the rear-spanning portion; a spectroscopic sensor 1909 which is configured to collect data concerning light energy which is transmitted through and/or reflected from the person's body, which is located on the inserted portion; a sound-emitting unit 1910, which is located on the inserted portion; a power source 1911; and a data processor 1912.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion; and rear-spanning portion. In an example, a microphone can be located on a portion of an ear attachment selected from the group consisting of: frontal-connecting portion; inserted portion; and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: inserted portion; and rear-spanning portion.

In an example, a sound-emitting unit can be a speaker. In an example, a sound-emitting unit can be located on a portion of an ear attachment selected from the group consisting of: inserted portion; frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 20 shows an example of how this invention can be embodied in a wearable device for the ear comprising: an ear attachment, wherein this ear attachment further comprises an inserted portion 2001 which is configured to be at least partially inserted into the ear canal, a frontal-connecting portion 2002 which is configured to span a portion of an outer ear or ear canal opening perimeter between 11 o'clock (or 330 degree) and 4 o'clock (or 120 degree) positions, a rear-spanning portion 2004 which is configured to span a portion of an outer ear or ear canal opening perimeter between 5 o'clock (or 150 degree) and 1 o'clock (or 30 degree) positions; an electromagnetic energy sensor 2006 which is configured to collect data concerning electromagnetic energy which is transmitted through and/or emitted by the person's body, which is located on the inserted portion; a microphone 2007, which is located on the rear-spanning portion; a sound-emitting unit 2010, which is located on the inserted portion; a power source 2011; and a data processor 2012.

In an example, an inserted portion of an ear attachment can be completely inserted into the ear canal or only partially inserted into the ear canal. In an example, a frontal-connecting portion of an ear attachment can be configured to span from the upper portion of the outer ear to an inserted portion of an ear attachment. In an example, a frontal-connecting portion of an ear attachment can be connected to (or integrated with) an inserted portion of an ear attachment. In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion; and rear-spanning portion.

In an example, a microphone can be located on a portion of an ear attachment selected from the group consisting of: frontal-connecting portion; inserted portion; and rear-spanning portion. In an example, a sound-emitting unit can be a speaker. In an example, a speaker can send a message into the person's ear (such as “Mar. 19, 1963 was a good day”). In an example, a sound-emitting unit can be located on a portion of an ear attachment selected from the group consisting of: inserted portion; frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

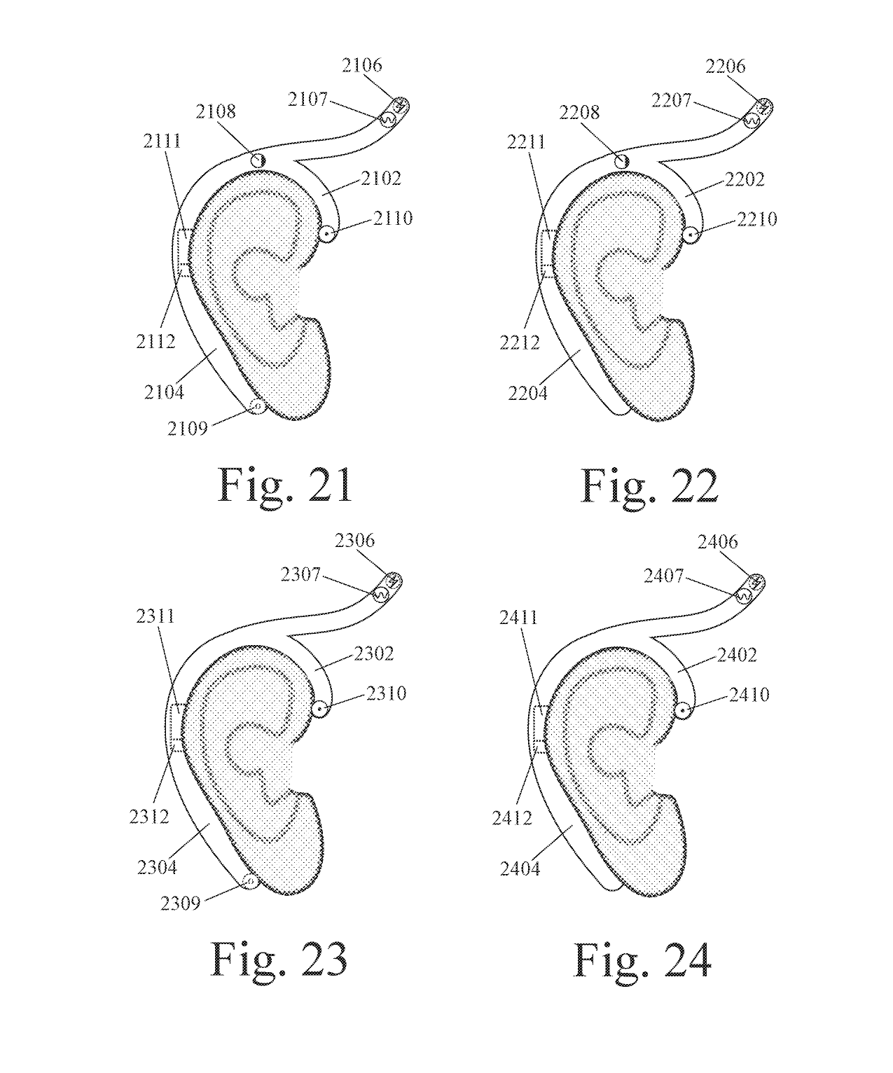

FIG. 21 shows an example of how this invention can be embodied in a wearable device for the ear comprising: an ear attachment, wherein this ear attachment further comprises a frontal-connecting portion 2102 which is configured to span a portion of an outer ear or ear canal opening perimeter between 11 o'clock (or 330 degree) and 4 o'clock (or 120 degree) positions, a forehead-projecting portion 2103 which is configured to span from the ear to the person's temple and/or forehead, a rear-spanning portion 2104 which is configured to span a portion of an outer ear or ear canal opening perimeter between 5 o'clock (or 150 degree) and 1 o'clock (or 30 degree) positions; an electromagnetic energy sensor 2106 which is configured to collect data concerning electromagnetic energy which is transmitted through and/or emitted by the person's body, which is located on the forehead-projecting portion; a microphone 2107, which is located on the forehead-projecting portion; a camera 2108, which is located on the frontal-connecting portion; a spectroscopic sensor 2109 which is configured to collect data concerning light energy which is transmitted through and/or reflected from the person's body, which is located on the rear-spanning portion; a sound-emitting unit 2110, which is located on the frontal-connecting portion; a power source 2111; and a data processor 2112.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; and rear-spanning portion. In an example, a microphone can be located on a portion of an ear attachment selected from the group consisting of: frontal-connecting portion; forehead-projecting portion; and rear-spanning portion. In an example, a camera can be located on a portion of an ear attachment selected from the group consisting of: frontal-connecting portion and forehead-projecting portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible.

In an example, a sound-emitting unit can be a speaker. In an example, a sound-emitting unit can be located on a portion of an ear attachment selected from the group consisting of: frontal-connecting portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 22 shows an example of how this invention can be embodied in a wearable device for the ear comprising: an ear attachment, wherein this ear attachment further comprises a frontal-connecting portion 2202 which is configured to span a portion of an outer ear or ear canal opening perimeter between 11 o'clock (or 330 degree) and 4 o'clock (or 120 degree) positions, a forehead-projecting portion 2203 which is configured to span from the ear to the person's temple and/or forehead, a rear-spanning portion 2204 which is configured to span a portion of an outer ear or ear canal opening perimeter between 5 o'clock (or 150 degree) and 1 o'clock (or 30 degree) positions; an electromagnetic energy sensor 2206 which is configured to collect data concerning electromagnetic energy which is transmitted through and/or emitted by the person's body, which is located on the forehead-projecting portion; a microphone 2207, which is located on the forehead-projecting portion; a camera 2208, which is located on the frontal-connecting portion; a sound-emitting unit 2210, which is located on the frontal-connecting portion; a power source 2211; and a data processor 2212.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion and rear-spanning portion.

In an example, a microphone can be located on a portion of an ear attachment selected from the group consisting of: frontal-connecting portion; forehead-projecting portion; and rear-spanning portion. In an example, a camera can be located on a portion of an ear attachment selected from the group consisting of: frontal-connecting portion and forehead-projecting portion. In an example, a sound-emitting unit can be a speaker. In an example, a sound-emitting unit can be located on a portion of an ear attachment selected from the group consisting of: frontal-connecting portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 23 shows an example of how this invention can be embodied in a wearable device for the ear comprising: an ear attachment, wherein this ear attachment further comprises a frontal-connecting portion 2302 which is configured to span a portion of an outer ear or ear canal opening perimeter between 11 o'clock (or 330 degree) and 4 o'clock (or 120 degree) positions, a forehead-projecting portion 2303 which is configured to span from the ear to the person's temple and/or forehead, a rear-spanning portion 2304 which is configured to span a portion of an outer ear or ear canal opening perimeter between 5 o'clock (or 150 degree) and 1 o'clock (or 30 degree) positions; an electromagnetic energy sensor 2306 which is configured to collect data concerning electromagnetic energy which is transmitted through and/or emitted by the person's body, which is located on the forehead-projecting portion; a microphone 2307, which is located on the forehead-projecting portion; a spectroscopic sensor 2309 which is configured to collect data concerning light energy which is transmitted through and/or reflected from the person's body, which is located on the rear-spanning portion; a sound-emitting unit 2310, which is located on the frontal-connecting portion; a power source 2311; and a data processor 2312.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion and rear-spanning portion.

In an example, a microphone can be located on a portion of an ear attachment selected from the group consisting of: frontal-connecting portion; forehead-projecting portion; and rear-spanning portion. In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible.

In an example, a sound-emitting unit can be a speaker. In an example, a sound-emitting unit can be located on a portion of an ear attachment selected from the group consisting of: frontal-connecting portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 24 shows an example of how this invention can be embodied in a wearable device for the ear comprising: an ear attachment, wherein this ear attachment further comprises a frontal-connecting portion 2402 which is configured to span a portion of an outer ear or ear canal opening perimeter between 11 o'clock (or 330 degree) and 4 o'clock (or 120 degree) positions, a forehead-projecting portion 2403 which is configured to span from the ear to the person's temple and/or forehead, a rear-spanning portion 2404 which is configured to span a portion of an outer ear or ear canal opening perimeter between 5 o'clock (or 150 degree) and 1 o'clock (or 30 degree) positions; an electromagnetic energy sensor 2406 which is configured to collect data concerning electromagnetic energy which is transmitted through and/or emitted by the person's body, which is located on the forehead-projecting portion; a microphone 2407, which is located on the forehead-projecting portion; a sound-emitting unit 2410, which is located on the frontal-connecting portion; a power source 2411; and a data processor 2412.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a forehead-projecting portion of an ear attachment can be configured to span from the upper portion of the outer ear to the person's temple and/or forehead. In an example, a forehead-projecting portion of an ear attachment can be connected to (or integrated with) a frontal-connecting portion of an ear attachment. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion and rear-spanning portion.

In an example, a microphone can be located on a portion of an ear attachment selected from the group consisting of: frontal-connecting portion; forehead-projecting portion; and rear-spanning portion. In an example, a sound-emitting unit can be a speaker. In an example, a sound-emitting unit can be located on a portion of an ear attachment selected from the group consisting of: frontal-connecting portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

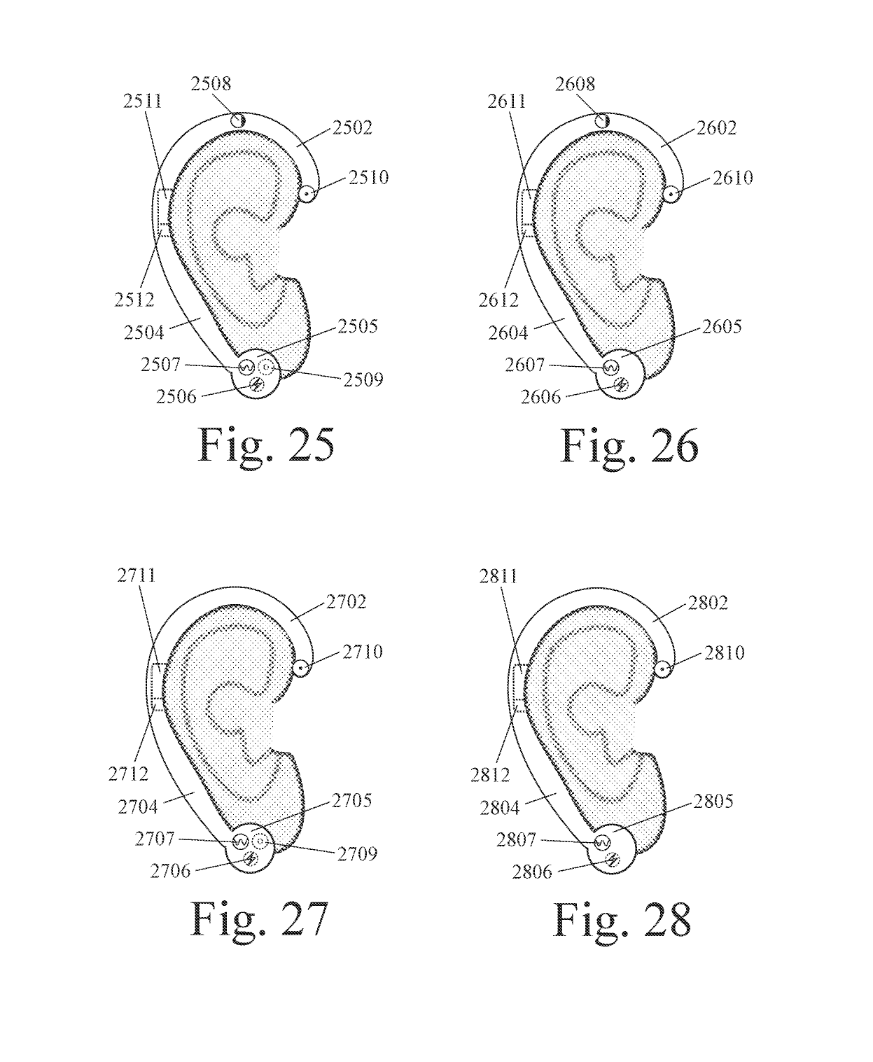

FIG. 25 shows an example of how this invention can be embodied in a wearable device for the ear comprising: an ear attachment, wherein this ear attachment further comprises a frontal-connecting portion 2502 which is configured to span a portion of an outer ear or ear canal opening perimeter between 11 o'clock (or 330 degree) and 4 o'clock (or 120 degree) positions, a rear-spanning portion 2504 which is configured to span a portion of an outer ear or ear canal opening perimeter between 5 o'clock (or 150 degree) and 1 o'clock (or 30 degree) positions, and an earlobe portion 2505 which is configured to be attached to the earlobe; an electromagnetic energy sensor 2506 which is configured to collect data concerning electromagnetic energy which is transmitted through and/or emitted by the person's body, which is located on the earlobe portion; a microphone 2507, which is located on the earlobe portion; a camera 2508, which is located on the frontal-connecting portion; a spectroscopic sensor 2509 which is configured to collect data concerning light energy which is transmitted through and/or reflected from the person's body, which is located on the earlobe portion; a sound-emitting unit 2510, which is located on the frontal-connecting portion; a power source 2511; and a data processor 2512.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: earlobe portion and rear-spanning portion. In an example, a microphone can be located on a portion of an ear attachment selected from the group consisting of: earlobe portion; frontal-connecting portion; and rear-spanning portion. In an example, a camera can be located on a portion of an ear attachment selected from the group consisting of: frontal-connecting portion; and earlobe portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion and rear-spanning portion.

In an example, a sound-emitting unit can be a speaker. In an example, a sound-emitting unit can be located on a portion of an ear attachment selected from the group consisting of: frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 26 shows an example of how this invention can be embodied in a wearable device for the ear comprising: an ear attachment, wherein this ear attachment further comprises a frontal-connecting portion 2602 which is configured to span a portion of an outer ear or ear canal opening perimeter between 11 o'clock (or 330 degree) and 4 o'clock (or 120 degree) positions, a rear-spanning portion 2604 which is configured to span a portion of an outer ear or ear canal opening perimeter between 5 o'clock (or 150 degree) and 1 o'clock (or 30 degree) positions, and an earlobe portion 2605 which is configured to be attached to the earlobe; an electromagnetic energy sensor 2606 which is configured to collect data concerning electromagnetic energy which is transmitted through and/or emitted by the person's body, which is located on the earlobe portion; a microphone 2607, which is located on the earlobe portion; a camera 2608, which is located on the frontal-connecting portion; a sound-emitting unit 2610, which is located on the frontal-connecting portion; a power source 2611; and a data processor 2612.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: earlobe portion and rear-spanning portion.

In an example, a microphone can be located on a portion of an ear attachment selected from the group consisting of: earlobe portion; frontal-connecting portion; and rear-spanning portion. In an example, a camera can be located on a portion of an ear attachment selected from the group consisting of: frontal-connecting portion; and earlobe portion. In an example, a sound-emitting unit can be a speaker. In an example, a sound-emitting unit can be located on a portion of an ear attachment selected from the group consisting of: frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 27 shows an example of how this invention can be embodied in a wearable device for the ear comprising: an ear attachment, wherein this ear attachment further comprises a frontal-connecting portion 2702 which is configured to span a portion of an outer ear or ear canal opening perimeter between 11 o'clock (or 330 degree) and 4 o'clock (or 120 degree) positions, a rear-spanning portion 2704 which is configured to span a portion of an outer ear or ear canal opening perimeter between 5 o'clock (or 150 degree) and 1 o'clock (or 30 degree) positions, and an earlobe portion 2705 which is configured to be attached to the earlobe; an electromagnetic energy sensor 2706 which is configured to collect data concerning electromagnetic energy which is transmitted through and/or emitted by the person's body, which is located on the earlobe portion; a microphone 2707, which is located on the earlobe portion; a spectroscopic sensor 2709 which is configured to collect data concerning light energy which is transmitted through and/or reflected from the person's body, which is located on the earlobe portion; a sound-emitting unit 2710, which is located on the frontal-connecting portion; a power source 2711; and a data processor 2712.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: earlobe portion and rear-spanning portion. In an example, a microphone can be located on a portion of an ear attachment selected from the group consisting of: earlobe portion; frontal-connecting portion; and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion and rear-spanning portion.

In an example, a sound-emitting unit can be a speaker. In an example, a sound-emitting unit can be located on a portion of an ear attachment selected from the group consisting of: frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 28 shows an example of how this invention can be embodied in a wearable device for the ear comprising: an ear attachment, wherein this ear attachment further comprises a frontal-connecting portion 2802 which is configured to span a portion of an outer ear or ear canal opening perimeter between 11 o'clock (or 330 degree) and 4 o'clock (or 120 degree) positions, a rear-spanning portion 2804 which is configured to span a portion of an outer ear or ear canal opening perimeter between 5 o'clock (or 150 degree) and 1 o'clock (or 30 degree) positions, and an earlobe portion 2805 which is configured to be attached to the earlobe; an electromagnetic energy sensor 2806 which is configured to collect data concerning electromagnetic energy which is transmitted through and/or emitted by the person's body, which is located on the earlobe portion; a microphone 2807, which is located on the earlobe portion; a sound-emitting unit 2810, which is located on the frontal-connecting portion; a power source 2811; and a data processor 2812.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: earlobe portion and rear-spanning portion.

In an example, a microphone can be located on a portion of an ear attachment selected from the group consisting of: earlobe portion; frontal-connecting portion; and rear-spanning portion. In an example, a sound-emitting unit can be a speaker. In an example, a sound-emitting unit can be located on a portion of an ear attachment selected from the group consisting of: frontal-connecting portion; earlobe portion; and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 29 shows an example of how this invention can be embodied in a wearable device for the ear comprising: an ear attachment, wherein this ear attachment further comprises a forehead-projecting portion 2903 which is configured to span from the ear to the person's temple and/or forehead, a rear-spanning portion 2904 which is configured to span a portion of an outer ear or ear canal opening perimeter between 5 o'clock (or 150 degree) and 1 o'clock (or 30 degree) positions, and an earlobe portion 2905 which is configured to be attached to the earlobe; an electromagnetic energy sensor 2906 which is configured to collect data concerning electromagnetic energy which is transmitted through and/or emitted by the person's body, which is located on the forehead-projecting portion; a microphone 2907, which is located on the earlobe portion; a camera 2908, which is located on the forehead-projecting portion; a spectroscopic sensor 2909 which is configured to collect data concerning light energy which is transmitted through and/or reflected from the person's body, which is located on the earlobe portion; a sound-emitting unit 2910, which is located on the rear-spanning portion; a power source 2911; and a data processor 2912.

In an example, a forehead-projecting portion of an ear attachment can be configured to span from the upper portion of the outer ear to the person's temple and/or forehead. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; earlobe portion; and rear-spanning portion. In an example, a microphone can be located on a portion of an ear attachment selected from the group consisting of: earlobe portion; forehead-projecting portion; and rear-spanning portion. In an example, a camera can be located on a portion of an ear attachment selected from the group consisting of: forehead-projecting portion and earlobe portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion; and rear-spanning portion.

In an example, a sound-emitting unit can be a speaker. In an example, a sound-emitting unit can be located on a portion of an ear attachment selected from the group consisting of: earlobe portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 30 shows an example of how this invention can be embodied in a wearable device for the ear comprising: an ear attachment, wherein this ear attachment further comprises a forehead-projecting portion 3003 which is configured to span from the ear to the person's temple and/or forehead, a rear-spanning portion 3004 which is configured to span a portion of an outer ear or ear canal opening perimeter between 5 o'clock (or 150 degree) and 1 o'clock (or 30 degree) positions, and an earlobe portion 3005 which is configured to be attached to the earlobe; an electromagnetic energy sensor 3006 which is configured to collect data concerning electromagnetic energy which is transmitted through and/or emitted by the person's body, which is located on the forehead-projecting portion; a microphone 3007, which is located on the earlobe portion; a camera 3008, which is located on the forehead-projecting portion; a sound-emitting unit 3010, which is located on the rear-spanning portion; a power source 3011; and a data processor 3012.

In an example, a forehead-projecting portion of an ear attachment can be configured to span from the upper portion of the outer ear to the person's temple and/or forehead. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; earlobe portion; and rear-spanning portion.

In an example, a microphone can be located on a portion of an ear attachment selected from the group consisting of: earlobe portion; forehead-projecting portion; and rear-spanning portion. In an example, a camera can be located on a portion of an ear attachment selected from the group consisting of: forehead-projecting portion and earlobe portion. In an example, a sound-emitting unit can be a speaker. In an example, a sound-emitting unit can be located on a portion of an ear attachment selected from the group consisting of: earlobe portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

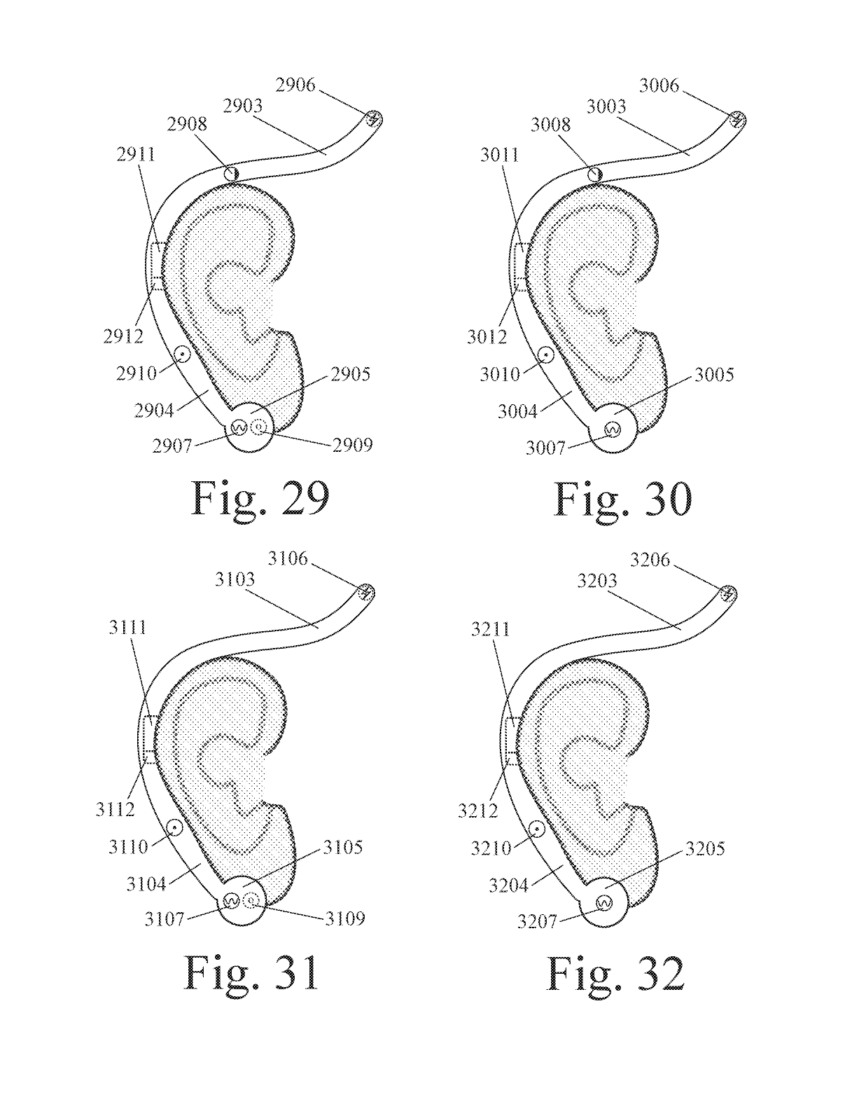

FIG. 31 shows an example of how this invention can be embodied in a wearable device for the ear comprising: an ear attachment, wherein this ear attachment further comprises a forehead-projecting portion 3103 which is configured to span from the ear to the person's temple and/or forehead, a rear-spanning portion 3104 which is configured to span a portion of an outer ear or ear canal opening perimeter between 5 o'clock (or 150 degree) and 1 o'clock (or 30 degree) positions, and an earlobe portion 3105 which is configured to be attached to the earlobe; an electromagnetic energy sensor 3106 which is configured to collect data concerning electromagnetic energy which is transmitted through and/or emitted by the person's body, which is located on the forehead-projecting portion; a microphone 3107, which is located on the earlobe portion; a spectroscopic sensor 3109 which is configured to collect data concerning light energy which is transmitted through and/or reflected from the person's body, which is located on the earlobe portion; a sound-emitting unit 3110, which is located on the rear-spanning portion; a power source 3111; and a data processor 3112.

In an example, a forehead-projecting portion of an ear attachment can be configured to span from the upper portion of the outer ear to the person's temple and/or forehead. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; earlobe portion; and rear-spanning portion. In an example, a microphone can be located on a portion of an ear attachment selected from the group consisting of: earlobe portion; forehead-projecting portion; and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion; and rear-spanning portion.

In an example, a sound-emitting unit can be a speaker. In an example, a sound-emitting unit can be located on a portion of an ear attachment selected from the group consisting of: earlobe portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 32 shows an example of how this invention can be embodied in a wearable device for the ear comprising: an ear attachment, wherein this ear attachment further comprises a forehead-projecting portion 3203 which is configured to span from the ear to the person's temple and/or forehead, a rear-spanning portion 3204 which is configured to span a portion of an outer ear or ear canal opening perimeter between 5 o'clock (or 150 degree) and 1 o'clock (or 30 degree) positions, and an earlobe portion 3205 which is configured to be attached to the earlobe; an electromagnetic energy sensor 3206 which is configured to collect data concerning electromagnetic energy which is transmitted through and/or emitted by the person's body, which is located on the forehead-projecting portion; a microphone 3207, which is located on the earlobe portion; a sound-emitting unit 3210, which is located on the rear-spanning portion; a power source 3211; and a data processor 3212.

In an example, a forehead-projecting portion of an ear attachment can be configured to span from the upper portion of the outer ear to the person's temple and/or forehead. In an example, a forehead-projecting portion can curve upwards as it projects forward from a person's ear. In an example, a forehead-projecting portion can curve toward the center of a person's forehead as it projects forward from a person's ear. In an example, a forehead-projecting portion can be configured with a spring, other tensile mechanism, or actuator such that its front portion presses gently against a person's temple and/or forehead. In an example, a forehead-projecting portion can be sinusoidal. In an example, a forehead-projecting portion can have a length between 1 and 5 inches.

In an example, a rear-spanning portion of an ear attachment can be configured to span from the rear portion of the outer ear to the upper portion of the outer ear. In an example, a rear-spanning portion of an ear attachment can be connected to (or integrated with) a forehead-projecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be connected to (or integrated with) a rear-spanning portion of an ear attachment. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: forehead-projecting portion; earlobe portion; and rear-spanning portion.

In an example, a microphone can be located on a portion of an ear attachment selected from the group consisting of: earlobe portion; forehead-projecting portion; and rear-spanning portion. In an example, a sound-emitting unit can be a speaker. In an example, a sound-emitting unit can be located on a portion of an ear attachment selected from the group consisting of: earlobe portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

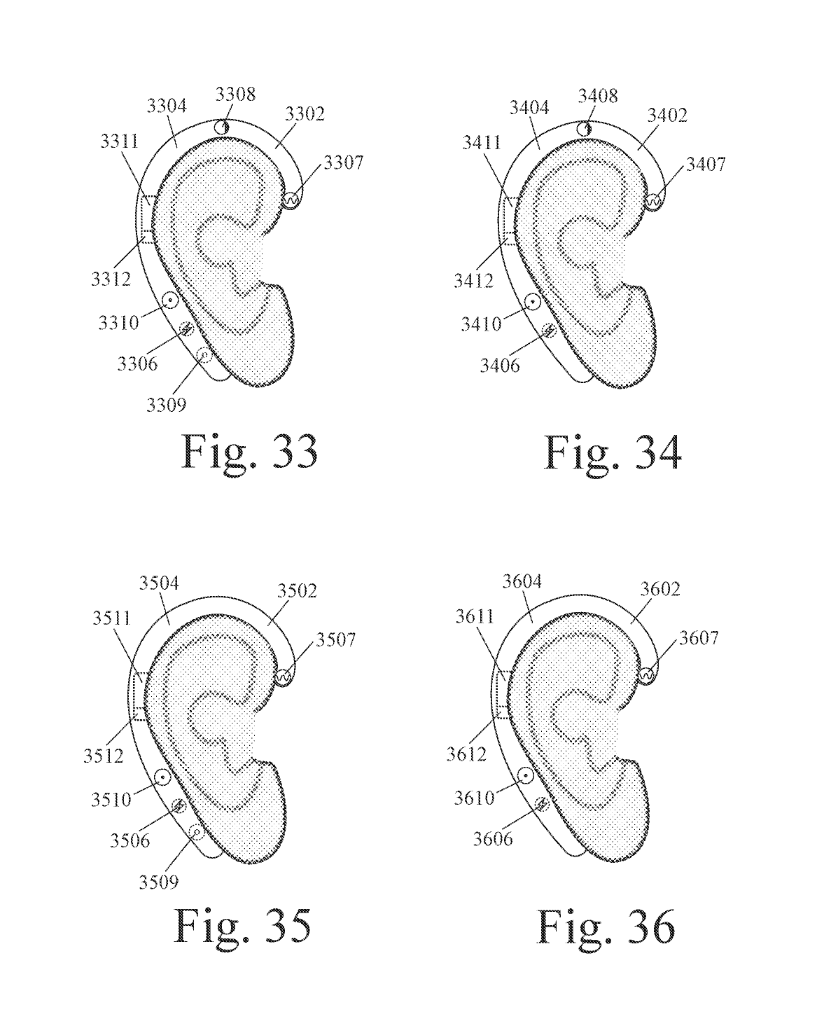

FIG. 33 shows an example of how this invention can be embodied in a wearable device for the ear comprising: an ear attachment, wherein this ear attachment further comprises a frontal-connecting portion 3302 which is configured to span a portion of an outer ear or ear canal opening perimeter between 11 o'clock (or 330 degree) and 4 o'clock (or 120 degree) positions, a rear-spanning portion 3304 which is configured to span a portion of an outer ear or ear canal opening perimeter between 5 o'clock (or 150 degree) and 1 o'clock (or 30 degree) positions; an electromagnetic energy sensor 3306 which is configured to collect data concerning electromagnetic energy which is transmitted through and/or emitted by the person's body, which is located on the rear-spanning portion; a microphone 3307, which is located on the frontal-connecting portion; a camera 3308, which is located on the frontal-connecting portion; a spectroscopic sensor 3309 which is configured to collect data concerning light energy which is transmitted through and/or reflected from the person's body, which is located on the rear-spanning portion; a sound-emitting unit 3310, which is located on the rear-spanning portion; a power source 3311; and a data processor 3312.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, a microphone can be located on a portion of an ear attachment selected from the group consisting of: frontal-connecting portion and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible.

In an example, a sound-emitting unit can be a speaker. In an example, a sound-emitting unit can be located on a portion of an ear attachment selected from the group consisting of: frontal-connecting portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 34 shows an example of how this invention can be embodied in a wearable device for the ear comprising: an ear attachment, wherein this ear attachment further comprises a frontal-connecting portion 3402 which is configured to span a portion of an outer ear or ear canal opening perimeter between 11 o'clock (or 330 degree) and 4 o'clock (or 120 degree) positions, a rear-spanning portion 3404 which is configured to span a portion of an outer ear or ear canal opening perimeter between 5 o'clock (or 150 degree) and 1 o'clock (or 30 degree) positions; an electromagnetic energy sensor 3406 which is configured to collect data concerning electromagnetic energy which is transmitted through and/or emitted by the person's body, which is located on the rear-spanning portion; a microphone 3407, which is located on the frontal-connecting portion; a camera 3408, which is located on the frontal-connecting portion; a sound-emitting unit 3410, which is located on the rear-spanning portion; a power source 3411; and a data processor 3412.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor.

In an example, a microphone can be located on a portion of an ear attachment selected from the group consisting of: frontal-connecting portion and rear-spanning portion. In an example, a sound-emitting unit can be a speaker. In an example, a sound-emitting unit can be located on a portion of an ear attachment selected from the group consisting of: frontal-connecting portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 35 shows an example of how this invention can be embodied in a wearable device for the ear comprising: an ear attachment, wherein this ear attachment further comprises a frontal-connecting portion 3502 which is configured to span a portion of an outer ear or ear canal opening perimeter between 11 o'clock (or 330 degree) and 4 o'clock (or 120 degree) positions, a rear-spanning portion 3504 which is configured to span a portion of an outer ear or ear canal opening perimeter between 5 o'clock (or 150 degree) and 1 o'clock (or 30 degree) positions; an electromagnetic energy sensor 3506 which is configured to collect data concerning electromagnetic energy which is transmitted through and/or emitted by the person's body, which is located on the rear-spanning portion; a microphone 3507, which is located on the frontal-connecting portion; a spectroscopic sensor 3509 which is configured to collect data concerning light energy which is transmitted through and/or reflected from the person's body, which is located on the rear-spanning portion; a sound-emitting unit 3510, which is located on the rear-spanning portion; a power source 3511; and a data processor 3512.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, a microphone can be located on a portion of an ear attachment selected from the group consisting of: frontal-connecting portion and rear-spanning portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible.

In an example, a sound-emitting unit can be a speaker. In an example, a sound-emitting unit can be located on a portion of an ear attachment selected from the group consisting of: frontal-connecting portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 36 shows an example of how this invention can be embodied in a wearable device for the ear comprising: an ear attachment, wherein this ear attachment further comprises a frontal-connecting portion 3602 which is configured to span a portion of an outer ear or ear canal opening perimeter between 11 o'clock (or 330 degree) and 4 o'clock (or 120 degree) positions, a rear-spanning portion 3604 which is configured to span a portion of an outer ear or ear canal opening perimeter between 5 o'clock (or 150 degree) and 1 o'clock (or 30 degree) positions; an electromagnetic energy sensor 3606 which is configured to collect data concerning electromagnetic energy which is transmitted through and/or emitted by the person's body, which is located on the rear-spanning portion; a microphone 3607, which is located on the frontal-connecting portion; a sound-emitting unit 3610, which is located on the rear-spanning portion; a power source 3611; and a data processor 3612.

In an example, a frontal-connecting portion can partially encircle the outer perimeter of an ear and/or the outer opening of the ear canal. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a frontal-connecting portion can span a portion of an outer ear or ear canal opening perimeter within a range selected from the group consisting of: 11 o'clock (330 degrees) to 1 o'clock (30 degrees); 12 o'clock (0 degrees) to 1 o'clock (30 degrees); and 12 o'clock (0 degrees) to 2 o'clock (60 degrees).

In an example, a rear-spanning portion of an ear attachment can be configured to span from the rear portion of the outer ear to the upper portion of the outer ear. In an example, a rear-spanning portion of an ear attachment can be connected to (or integrated with) a frontal-connecting portion of an ear attachment. In an example, a rear-spanning portion can partially encircle the outer perimeter of an ear. Radial clock hour (or degree) positions around the perimeter of an outer ear or ear canal opening can be defined as shown in FIG. 47, wherein the 12 o'clock (or 0 degree) position is vertically above the center of the outer opening of the ear canal. In an example, a rear-spanning portion can span a portion of an outer ear or ear canal opening perimeter within a range selected from the group consisting of: 6 o'clock (180 degrees) to 12 o'clock (0 degrees); 8 o'clock (240 degrees) to 1 o'clock (30 degrees); 8 o'clock (240 degrees) to 11 o'clock (330 degrees); 8 o'clock (240 degrees) to 12 o'clock (0 degrees); and 9 o'clock (270 degrees) to 12 o'clock (0 degrees).

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor.

In an example, a microphone can be located on a portion of an ear attachment selected from the group consisting of: frontal-connecting portion and rear-spanning portion. In an example, a sound-emitting unit can be a speaker. In an example, a sound-emitting unit can be located on a portion of an ear attachment selected from the group consisting of: frontal-connecting portion and rear-spanning portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

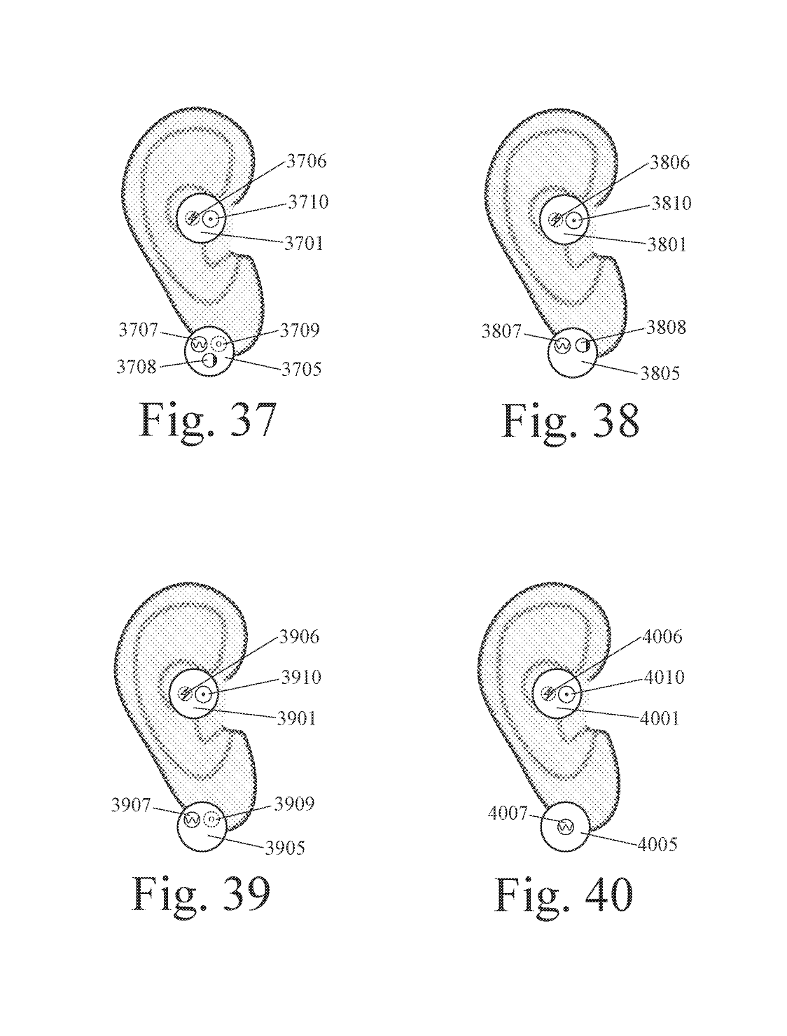

FIG. 37 shows an example of how this invention can be embodied in a wearable device for the ear comprising: an ear attachment, wherein this ear attachment further comprises an inserted portion 3701 which is configured to be at least partially inserted into the ear canal, and an earlobe portion 3705 which is configured to be attached to the earlobe; an electromagnetic energy sensor 3706 which is configured to collect data concerning electromagnetic energy which is transmitted through and/or emitted by the person's body, which is located on the inserted portion; a microphone 3707, which is located on the earlobe portion; a camera 3708, which is located on the earlobe portion; a spectroscopic sensor 3709 which is configured to collect data concerning light energy which is transmitted through and/or reflected from the person's body, which is located on the earlobe portion; and a sound-emitting unit 3710, which is located on the inserted portion.

In an example, an inserted portion of an ear attachment can be completely inserted into the ear canal or only partially inserted into the ear canal. In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion and earlobe portion. In an example, a microphone can be located on a portion of an ear attachment selected from the group consisting of: earlobe portion and inserted portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion and inserted portion.

In an example, a sound-emitting unit can be a speaker. In an example, a sound-emitting unit can be located on a portion of an ear attachment selected from the group consisting of: inserted portion and earlobe portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 38 shows an example of how this invention can be embodied in a wearable device for the ear comprising: an ear attachment, wherein this ear attachment further comprises an inserted portion 3801 which is configured to be at least partially inserted into the ear canal, and an earlobe portion 3805 which is configured to be attached to the earlobe; an electromagnetic energy sensor 3806 which is configured to collect data concerning electromagnetic energy which is transmitted through and/or emitted by the person's body, which is located on the inserted portion; a microphone 3807, which is located on the earlobe portion; a camera 3808, which is located on the earlobe portion; and a sound-emitting unit 3810, which is located on the inserted portion.

In an example, an inserted portion of an ear attachment can be completely inserted into the ear canal or only partially inserted into the ear canal. In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion and earlobe portion.

In an example, a microphone can be located on a portion of an ear attachment selected from the group consisting of: earlobe portion and inserted portion. In an example, a sound-emitting unit can be a speaker. In an example, a sound-emitting unit can be located on a portion of an ear attachment selected from the group consisting of: inserted portion and earlobe portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 39 shows an example of how this invention can be embodied in a wearable device for the ear comprising: an ear attachment, wherein this ear attachment further comprises an inserted portion 3901 which is configured to be at least partially inserted into the ear canal, and an earlobe portion 3905 which is configured to be attached to the earlobe; an electromagnetic energy sensor 3906 which is configured to collect data concerning electromagnetic energy which is transmitted through and/or emitted by the person's body, which is located on the inserted portion; a microphone 3907, which is located on the earlobe portion; a spectroscopic sensor 3909 which is configured to collect data concerning light energy which is transmitted through and/or reflected from the person's body, which is located on the earlobe portion; and a sound-emitting unit 3910, which is located on the inserted portion.

In an example, an inserted portion of an ear attachment can be completely inserted into the ear canal or only partially inserted into the ear canal. In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion and earlobe portion. In an example, a microphone can be located on a portion of an ear attachment selected from the group consisting of: earlobe portion and inserted portion.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible. In an example, a spectroscopic sensor can be located on a portion of an ear attachment selected from the group consisting of: earlobe portion and inserted portion.

In an example, a sound-emitting unit can be a speaker. In an example, a sound-emitting unit can be located on a portion of an ear attachment selected from the group consisting of: inserted portion and earlobe portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 40 shows an example of how this invention can be embodied in a wearable device for the ear comprising: an ear attachment, wherein this ear attachment further comprises an inserted portion 4001 which is configured to be at least partially inserted into the ear canal, and an earlobe portion 4005 which is configured to be attached to the earlobe; an electromagnetic energy sensor 4006 which is configured to collect data concerning electromagnetic energy which is transmitted through and/or emitted by the person's body, which is located on the inserted portion; a microphone 4007, which is located on the earlobe portion; and a sound-emitting unit 4010, which is located on the inserted portion.

In an example, an inserted portion of an ear attachment can be completely inserted into the ear canal or only partially inserted into the ear canal. In an example, an earlobe portion of an ear attachment can be attached to an earlobe by a method selected from the group consisting of: clipping, clamping, snapping, magnetism, adhering, hooking, and inserting. In an example, an earlobe portion of an ear attachment can be an ear ring. In an example, an earlobe portion of an ear attachment can further comprise one or more decorative members such as gems, crystals, or stones.

In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, an electromagnetic energy sensor can be located on a portion of an ear attachment selected from the group consisting of: inserted portion and earlobe portion.

In an example, a microphone can be located on a portion of an ear attachment selected from the group consisting of: earlobe portion and inserted portion. In an example, a sound-emitting unit can be a speaker. In an example, a sound-emitting unit can be located on a portion of an ear attachment selected from the group consisting of: inserted portion and earlobe portion. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

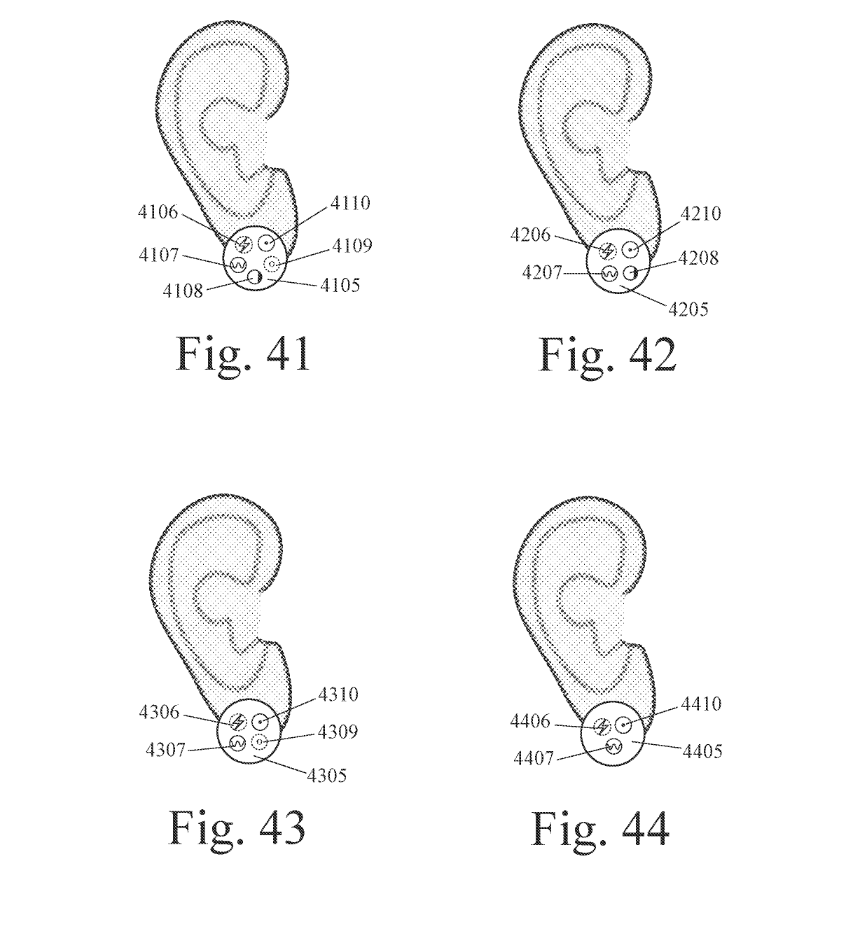

FIG. 41 shows an example of how this invention can be embodied in a wearable device for the ear comprising: an earlobe attachment 4105 which is configured to be attached to the earlobe; an electromagnetic energy sensor 4106 which is configured to collect data concerning electromagnetic energy which is transmitted through and/or emitted by the person's body; a microphone 4107; a camera 4108; a spectroscopic sensor 4109 which is configured to collect data concerning light energy which is transmitted through and/or reflected from the person's body; and a sound-emitting unit 4110.

In an example, an earlobe attachment can be attached to an earlobe by a method selected from the group consisting of: clipping, clamping, snapping, magnetism, adhering, hooking, and inserting. In an example, an earlobe attachment can be an ear ring. In an example, an earlobe attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible.

In an example, a sound-emitting unit can be a speaker. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 42 shows an example of how this invention can be embodied in a wearable device for the ear comprising: an earlobe attachment 4205 which is configured to be attached to the earlobe; an electromagnetic energy sensor 4206 which is configured to collect data concerning electromagnetic energy which is transmitted through and/or emitted by the person's body; a microphone 4207; a camera 4208; and a sound-emitting unit 4210.

In an example, an earlobe attachment can be attached to an earlobe by a method selected from the group consisting of: clipping, clamping, snapping, magnetism, adhering, hooking, and inserting. In an example, an earlobe attachment can be an ear ring. In an example, an earlobe attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor. In an example, a sound-emitting unit can be a speaker. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 43 shows an example of how this invention can be embodied in a wearable device for the ear comprising: earlobe attachment 4305 which is configured to be attached to the earlobe; an electromagnetic energy sensor 4306 which is configured to collect data concerning electromagnetic energy which is transmitted through and/or emitted by the person's body; a microphone 4307; a spectroscopic sensor 4309 which is configured to collect data concerning light energy which is transmitted through and/or reflected from the person's body; and a sound-emitting unit 4310.

In an example, an earlobe attachment can be attached to an earlobe by a method selected from the group consisting of: clipping, clamping, snapping, magnetism, adhering, hooking, and inserting. In an example, an earlobe attachment can be an ear ring. In an example, an earlobe attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible.

In an example, a sound-emitting unit can be a speaker. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 44 shows an example of how this invention can be embodied in a wearable device for the ear comprising: an earlobe attachment 4405 which is configured to be attached to the earlobe; an electromagnetic energy sensor 4406 which is configured to collect data concerning electromagnetic energy which is transmitted through and/or emitted by the person's body; a microphone 4407; and a sound-emitting unit 4410.

In an example, an earlobe attachment can be attached to an earlobe by a method selected from the group consisting of: clipping, clamping, snapping, magnetism, adhering, hooking, and inserting. In an example, an earlobe attachment can be an ear ring. In an example, an earlobe attachment can further comprise one or more decorative members such as gems, crystals, or stones. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor.

In an example, a sound-emitting unit can be a speaker. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

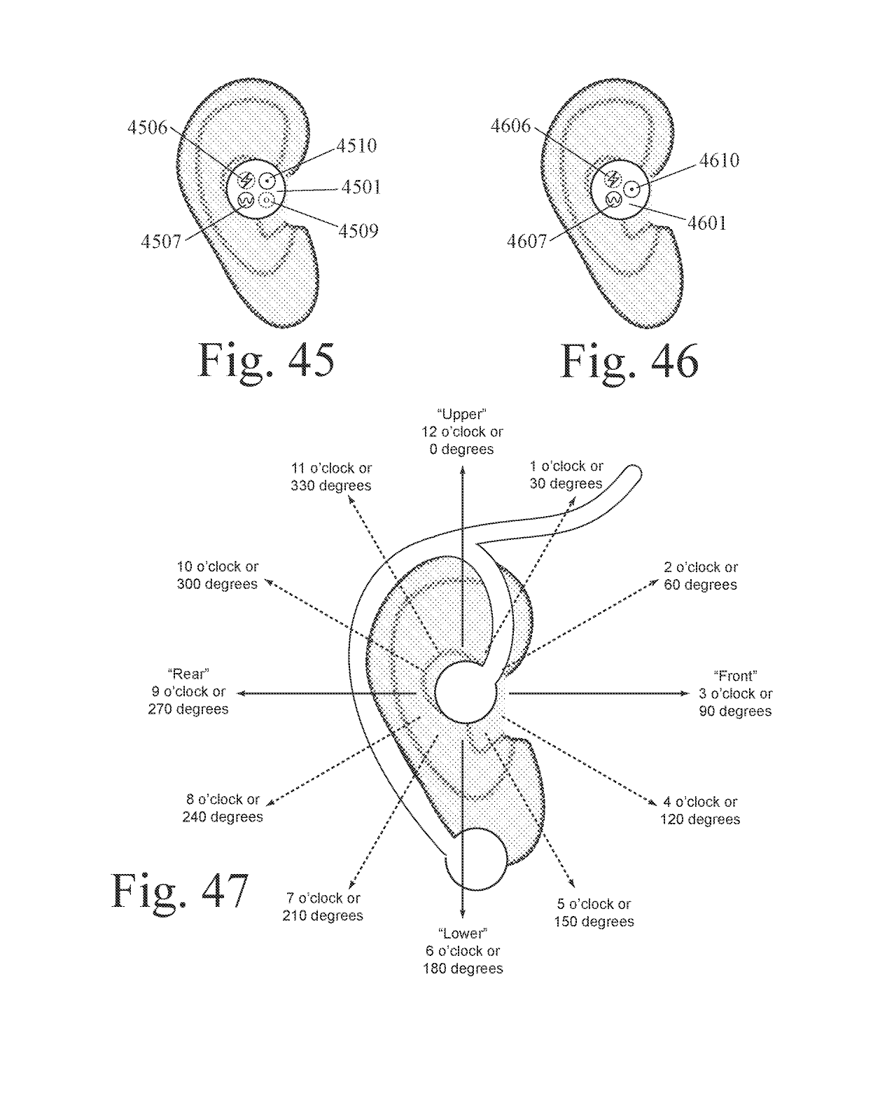

FIG. 45 shows an example of how this invention can be embodied in a wearable device for the ear comprising: an ear insert 4501 which is configured to be at least partially inserted into the ear canal; an electromagnetic energy sensor 4506 which is configured to collect data concerning electromagnetic energy which is transmitted through and/or emitted by the person's body; a microphone 4507; a spectroscopic sensor 4509 which is configured to collect data concerning light energy which is transmitted through and/or reflected from the person's body; and a sound-emitting unit 4510.

In an example, an inserted portion of an ear attachment can be completely inserted into the ear canal or only partially inserted into the ear canal. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor.

In an example, a spectroscopic sensor can collect data concerning light energy which is transmitted through a portion of the person's body. In an example, a spectroscopic sensor can collect data concerning light energy which is reflected from the person's body. In an example, this device can further comprise a light energy emitter which sends light energy toward the person's body. In an example, a spectroscopic sensor can collect data concerning light from a light energy emitter which has been transmitted through or reflected from a person's body. In an example, a spectroscopic sensor can collect data concerning ambient light which has been transmitted through or reflected from a person's body. In an example, data from a spectroscopic sensor can be analyzed to determine the spectral distribution of light which has been transmitted through or reflected from a person's body. In an example, a spectroscopic sensor can collect data concerning light in a portion of the spectrum selected from the group consisting of: infrared; near-infrared; ultraviolet; and visible.

In an example, a sound-emitting unit can be a speaker. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 46 shows an example of how this invention can be embodied in a wearable device for the ear comprising: an ear insert 4601 which is configured to be at least partially inserted into the ear canal; an electromagnetic energy sensor 4606 which is configured to collect data concerning electromagnetic energy which is transmitted through and/or emitted by the person's body; a microphone 4607; and a sound-emitting unit 4610.

In an example, an inserted portion of an ear attachment can be completely inserted into the ear canal or only partially inserted into the ear canal. In an example, an electromagnetic energy sensor can collect data concerning electromagnetic energy which is naturally emitted by the person's body. In an example, this device can further comprise an electromagnetic energy emitter which sends electromagnetic energy into the person's body. In an example, an electromagnetic energy sensor can measure electromagnetic energy which has been transmitted through the person's body. In an example, an electromagnetic energy sensor can collect data concerning the electromagnetic activity of the person's brain. In an example, an electromagnetic energy sensor can be an EEG or electroencephalographic sensor.

In an example, a sound-emitting unit can be a speaker. In an example, a power source can be a battery and/or an energy harvesting unit which transduces kinetic, light, thermal, or electromagnetic energy. In an example, a data processor can be a microprocessor or computer chip. In an example, this device can further comprise one or more components selected from the group consisting of: wireless data transmitter; wireless data receiver; LED or other light display; electromagnetic actuator; and button or other touch-based human-to-computer interface. Relevant variations discussed elsewhere in this disclosure or incorporated disclosures can also be applied to this example.

FIG. 47 illustrates how radial clock hour (or degree) positions can be defined for the perimeter of an outer ear or ear canal outer opening. The 12 o'clock (or 0 degree) position is the position on a perimeter which is intersected by a vertical line upwards from the center of the ear canal outer opening (when the person is standing upright). The 3 o'clock (or 90 degree) position is the position on a perimeter which is intersected by a horizontal vertical line forward from the center of the ear canal outer opening (when the person is standing upright). The 6 o'clock (or 180 degree) position is the position on a perimeter which is intersected by a vertical line downwards from the center of the ear canal outer opening (when the person is standing upright). The 9 o'clock (or 270 degree) position is the position on a perimeter which is intersected by a horizontal vertical line backward from the center of the ear canal outer opening (when the person is standing upright). Intermediate clock hour (or degree) positions are located between these four radial positions, as shown in FIG. 47.

I claim:

1. A wearable device for the ear comprising:
an ear attachment, wherein this ear attachment further comprises
an inserted portion which is configured to be at least partially inserted into the ear canal,
a frontal-connecting portion which is configured to span a portion of an outer ear or ear canal opening perimeter between the 11 o'clock and 4 o'clock positions, wherein clock hour positions around the perimeter of an outer ear or ear canal opening are defined relative to a 12 o'clock position that is vertically above the center of the outer opening of the ear canal,
a forehead-projecting portion which is configured to span from the ear to the person's temple and/or forehead,
a rear-spanning portion which is configured to span a portion of an outer ear or ear canal opening perimeter between the 5 o'clock and 1 o'clock positions, and
an earlobe portion which is configured to be attached to the earlobe;
an electromagnetic energy sensor which is configured to collect data concerning electromagnetic energy which is transmitted through and/or emitted by the person's body;
a microphone;
a spectroscopic sensor which is configured to collect data concerning light energy which is transmitted through and/or reflected from the person's body;
a sound-emitting unit;
a power source; and
a data processor.

2. The device in claim 1 wherein the forehead-projecting portion is connected to or integrated with the frontal-connecting portion.

3. The device in claim 1 wherein the forehead-projecting portion is configured to curve toward the center of a person's forehead as it projects forward from the person's ear.

4. The device in claim 1 wherein the forehead-projecting portion is configured with a tensile mechanism or actuator such that its front portion presses gently against a person's temple and/or forehead.

5. The device in claim 1 wherein the electromagnetic energy sensor is an electroencephalographic sensor.

6. The device in claim 1 wherein the electromagnetic energy sensor is located on the forehead-projecting portion.

7. The device in claim 1 wherein the electromagnetic energy sensor is located on the inserted portion.

8. The device in claim 1 wherein the electromagnetic energy sensor is located on the earlobe portion.

9. The device in claim 1 wherein the spectroscopic sensor is located on the earlobe portion.

10. A wearable device for the ear comprising:
an ear attachment, wherein this ear attachment further comprises
a frontal-connecting portion which is configured to span a portion of an outer ear or ear canal opening perimeter between the 11 o'clock and 4 o'clock positions, wherein clock hour positions around the perimeter of an outer ear or ear canal opening are defined relative to a 12 o'clock position that is vertically above the center of the outer opening of the ear canal,
a forehead-projecting portion which is configured to span from the ear to the person's temple and/or forehead,
a rear-spanning portion which is configured to span a portion of an outer ear or ear canal opening perimeter between the 5 o'clock and 1 o'clock positions, and
an earlobe portion which is configured to be attached to the earlobe;
an electromagnetic energy sensor which is configured to collect data concerning electromagnetic energy which is transmitted through and/or emitted by the person's body;
a microphone;
a spectroscopic sensor which is configured to collect data concerning light energy which is transmitted through and/or reflected from the person's body;
a sound-emitting unit;
a power source; and
a data processor.

11. The device in claim 10 wherein the forehead-projecting portion is connected to or integrated with the frontal-connecting portion.

12. The device in claim 10 wherein the forehead-projecting portion is configured to curve toward the center of a person's forehead as it projects forward from the person's ear.

13. The device in claim 10 wherein the forehead-projecting portion is configured with a tensile mechanism or actuator such that its front portion presses gently against a person's temple and/or forehead.

14. The device in claim 10 wherein the electromagnetic energy sensor is an electroencephalographic sensor.

15. The device in claim 10 wherein the electromagnetic energy sensor is located on the forehead-projecting portion.

16. The device in claim 10 wherein the electromagnetic energy sensor is located on the earlobe portion.

17. The device in claim 10 wherein the spectroscopic sensor is located on the earlobe portion.

18. A wearable device for the ear comprising:
an ear attachment, wherein this ear attachment further comprises
an inserted portion which is configured to be at least partially inserted into the ear canal,
a frontal-connecting portion which is configured to span a portion of an outer ear or ear canal opening perimeter between the 11 o'clock and 4 o'clock positions, wherein clock hour positions around the perimeter of an outer ear or ear canal opening are defined relative to a 12 o'clock position that is vertically above the center of the outer opening of the ear canal,
a rear-spanning portion which is configured to span a portion of an outer ear or ear canal opening perimeter between the 5 o'clock and 1 o'clock positions, and
an earlobe portion which is configured to be attached to the earlobe;
an electromagnetic energy sensor which is configured to collect data concerning electromagnetic energy which is transmitted through and/or emitted by the person's body;
a microphone;
a spectroscopic sensor which is configured to collect data concerning light energy which is transmitted through and/or reflected from the person's body;
a sound-emitting unit;
a power source; and
a data processor.

19. The device in claim 18 wherein the electromagnetic energy sensor is located on the inserted portion.

20. The device in claim 18 wherein the spectroscopic sensor is located on the earlobe portion.
